SEQUENCE LISTING (37 sequences)

SEQ ID NO: 1 - DNA, 44 bp, Artificial Sequence
  RT-primer sequence of the molecular marker miR-21
gatgaggagtgtcgtggagtcggcaatttcctcatcatcaacat

SEQ ID NO: 2 - DNA, 26 bp, Artificial Sequence
  PCR forward primer sequence of the miR-21
ctccgtcagggtagcttatcagactg

SEQ ID NO: 3 - DNA, 21 bp, Artificial Sequence
  PCR universal reverse primer sequence of miR-21
ctcaagtgtcgtggagtcggc

SEQ ID NO: 4 - DNA, 18 bp, Artificial Sequence
  probe sequence of miR-21
  misc_feature: 5′-FAM; MGB-3′
tttcctcatcatcaacat

SEQ ID NO: 5 - DNA, 44 bp, Artificial Sequence
  RT-primer sequence of the molecular marker miR-486-
gatgaggagtgtcgtggagtcggcaatttcctcatcactcgggg

SEQ ID NO: 6 - DNA, 26 bp, Artificial Sequence
  PCR forward primer sequence of miR-486-5p
ctccgtcagggtcctgtactgagctg

SEQ ID NO: 7 - DNA, 18 bp, Artificial Sequence
  specific probe sequence of miR-486-5p
tttcctcatcactcgggg

SEQ ID NO: 8 - DNA, 44 bp, Artificial Sequence
  RT-primer sequence of the molecular marker miR-205
gatgaggagtgtcgtggagtcggcaatttcctcatcacagactc

SEQ ID NO: 9 - DNA, 26 bp, Artificial Sequence
  PCR forward primer sequence of miR-205
ctccgtcagggtccttcattccaccg

SEQ ID NO: 10 - DNA, 18 bp, Artificial Sequence
  specific probe sequence of miR-205
tttcctcatcacagactc

SEQ ID NO: 11 - DNA, 44 bp, Artificial Sequence
  RT-primer sequence of the molecular marker miR-126
gatgaggagtgtcgtggagtcggcaatttcctcatcacgcatta

SEQ ID NO: 12 - DNA, 26 bp, Artificial Sequence
  PCR forward primer sequence of miR-126
ctccgtcagggtcgtaccgtgagtaa

SEQ ID NO: 13 - DNA, 18 bp, Artificial Sequence
  specific probe sequence of miR-126
tttcctcatcacgcatta

SEQ ID NO: 14 - DNA, 44 bp, Artificial Sequence
  RT-primer sequence of the molecular marker let-7a
gatgaggagtgtcgtggagtcggcaatttcctcatcaactatac

SEQ ID NO: 15 - DNA, 25 bp, Artificial Sequence
  PCR forward primer sequence of let-7a
ctccgtcagggtgaggtagtaggtt

SEQ ID NO: 16 - DNA, 18 bp, Artificial Sequence
  specific probe sequence of let-7a
tttcctcatcaactatac

SEQ ID NO: 17 - DNA, 45 bp, Artificial Sequence
  RT-primer sequence of the molecular marker miR-152
gatgaggagtgtcgtggagtcggcaatttcctcatcaagtcggag

SEQ ID NO: 18 - DNA, 26 bp, Artificial Sequence
  PCR forward primer sequence of miR-152
ctccgtcagggaggttctgtgataca

SEQ ID NO: 19 - DNA, 19 bp, Artificial Sequence
  specific probe sequence of miR-152
tttcctcatcaagtcggag

SEQ ID NO: 20 - DNA, 45 bp, Artificial Sequence; identical to SEQ ID NO: 17
  RT-primer sequence of the molecular marker miR-148a

SEQ ID NO: 21 - DNA, 25 bp, Artificial Sequence
  PCR forward primer sequence of miR-148a
ctccgtcagggaaagttctgagaca

SEQ ID NO: 22 - DNA, 19 bp, Artificial Sequence
  specific probe sequence of miR-148a
tttcctcatcaagtcggag

SEQ ID NO: 23 - DNA, 52 bp, Artificial Sequence
  first amplification template sequence of the molecular marker miR-21
gtcatcgcagacaacctcatctagactcatcaacatcagtctgataagctaa

SEQ ID NO: 24 - DNA, 52 bp, Artificial Sequence
  second amplification template sequence of miR-21
atcaacatcagtctgataagctaatctagactcgtcatcgcagacaacctca

SEQ ID NO: 25 - DNA, 40 bp, Artificial Sequence
  hybridization probe sequence of miR-21
agcctatcaacatcagtctgataagctaataggctgcatc

SEQ ID NO: 26 - DNA, 51 bp, Artificial Sequence
  first amplification template sequence of the molecular marker miR-486-5p
gtcatcgcagtgttcctcaacagactctctcggggcagctcagtacaggaa

SEQ ID NO: 27 - DNA, 52 bp, Artificial Sequence
  second amplification template sequence of miR-486-5p
ctcggggcagctcagtacaggaaaacagactcagtcatcgcagtgttcctca

SEQ ID NO: 28 - DNA, 41 bp, Artificial Sequence
  the hybridization probe sequence of miR-486-5p
agcctaactcggggcagctcagtacaggaataggctgcatc

SEQ ID NO: 29 - DNA, 51 bp, Artificial Sequence
  first amplification template sequence of the molecular marker miR-205
gtcatcgcagtgttcctcaacagactctcagactccggtggaatgaaggaa

SEQ ID NO: 30 - DNA, 51 bp, Artificial Sequence
  second amplification template sequence of miR-205
cagactccggtggaatgaaggaaacagactcagtcatcgcagtgttcctca

SEQ ID NO: 31 - DNA, 41 bp, Artificial Sequence
  hybridization probe sequence of miR-205
agcctaacagactccggtggaatgaaggaataggctgcatc

SEQ ID NO: 32 - DNA, 51 bp, Artificial Sequence
  first amplification template sequence of the molecular marker miR-126
gtcatcgcagtgttcctcaacagactctcgcattattactcacggtacgaa

SEQ ID NO: 33 - DNA, 51 bp, Artificial Sequence
  second amplification template sequence of miR-126
cgcattattactcacggtacgaaacagactcagtcatcgcagtgttcctca

SEQ ID NO: 34 - DNA, 41 bp, Artificial Sequence
  hybridization probe sequence of miR-126
agcctaacgcattattactcacggtacgaataggctgcatc

SEQ ID NO: 35 - DNA, 50 bp, Artificial Sequence
  first amplification template sequence of the Internal control gene Let-7a
gtcatcgcagtgttcctcaacagactctaactatacaacctactacctca

SEQ ID NO: 36 - DNA, 51 bp, Artificial Sequence
  second amplification template sequence of Let-7a
aactatacaacctactacctcaaacagactcagtcatcgcagtgttcctca

SEQ ID NO: 37 - DNA, 40 bp, Artificial Sequence
  hybridization probe sequence of Let-7a
agcctaaactatacaacctactacctcaataggctgcatc

(19) United States
(12) Patent Application Publication
(10) Pub. No.: US 2022/0136056 A1
(43) Pub. Date: May 5, 2022

(54) Application Of Lung Cancer-Associated MicroRNA Molecular Marker In Serum Exosome And Detection Kit Using Same

(71) Applicant: Jiangsu Microdiag Biomedicine Technology Co. Ltd, Jiangsu (CN)

(72) Inventors: Tao Wang, Suzhou (CN); XiangYun Qu, Suzhou (CN); ZhaoNan Dong, Suzhou (CN); YunLi Jia, Suzhou (CN); XueQing Ma, Suzhou (CN)

(73) Assignee: Jiangsu Microdiag Biomedicine Technology Co. Ltd, Jiangsu (CN)

(21) Appl. No.: 16/639,103

(22) PCT Filed: Jun. 6, 2018
(86) PCT No.: PCT/CN2018/090043

§ 371 Date: Feb. 13, 2020

(30) Foreign Application Priority Data
Aug. 14, 2017 (CN) 201710693028.1

(51) Int. Cl. C12Q 1/6886; C12Q 1/686

(52) U.S. Cl. CPC C12Q 1/6886; C12Q 2600/118; C12Q 2600/178; C12Q 1/686

(57) ABSTRACT

The present invention discloses an application of an exosome-related microRNA in preparing a marker or diagnosis and prediction of lung cancer and a kit, and develops a PCR platform-based two-step detection kit for miRNA, which has the outstanding advantage in the aspect of auxiliary diagnosis of lung cancer by the combined use of up-regulated and down-regulated molecular markers, and has the sensitivity as low as lcopy/μL, greatly improving the detection accuracy. In addition, the isothermal amplification-based one-step detection system for miRNA developed by the present invention is faster and more convenient, which can greatly improve detection efficiency and reduce detection costs.

### TECHNICAL FIELD

The present invention belongs to the technical field of medical molecular biology, and particularly relates to an application of an exosome miRNA molecular marker for lung cancer and a kit thereof.

### BACKGROUND

Exosome is a small membranous vesicle that can be widely distributed in various body fluids and secreted by many cells, has a diameter between 30 nm and 120 nm in general, contains cell-specific protein, lipid, and nucleic acid, and plays an important role in many physiological and pathological processes in addition to being able to carry and transfer important signal molecules to form a brand-new intercellular information transfer system and then change the functions of other cells. Researches show that the molecular characteristics of tumor exosome partially reflect the phenotype of the tumor from which it is derived, and the carried tumor-specific microRNAs and antigens can be used as tumor diagnosis markers. In addition, the exosome can selectively remove certain cellular proteins and transfer many types of molecules between cells, can induce and enhance the body immune reaction, and can play an important role in many physiological and pathological processes such as immune surveillance, inflammatory response, cancer occurrence and development, etc. In clinical cases of a variety of tumors, comprising bladder cancer, brain tumor, colorectal cancer and melanoma, the exosome can be separated from the serum or urine and other body fluid of a patient for early clinical diagnosis, or also for clinical risk or therapeutic effect evaluation and prognosis determination of tumors.

Exosome contains a large number of mRNAs and microRNAs, which not only can protect the stable existence of RNA in vitro from degradation, but also can serve as an effective carrier to transport RNA to specific target cells to play an important regulation role. More than 120 microRNAs carried by Exosome have multiple functions. For example, miR-1, miR-17, miR-18, miR-181 and miR-375 are related to angiogenesis, hemutopoiesis,extracellular secretion and tumor occurrence.

MicroRNA (also called miRNA or miR)-as the first place in the top ten scientific breakthroughs of Science in 2002-one of the major discoveries of life science research in the 21st century, plays a very important role in the regulation of biological developmental time and sequence and the occurrence of diseases. By regulating the expression of oncogenes and tumor suppressor genes, miRNA regulates cell differentiation, proliferation and apoptosis, thereby promoting or inhibiting tumor occurrence. During this period, there are complex regulation mechanisms that form a regulation network to jointly promote or inhibit tumor occurrence. Methylation, defects in biological origin, variation, abnormal transcription, and loss or amplification of the genome all lead to the abnormality of miRNA in human tumor. Many miRNAs directly act as a proto-oncogene or a tumor suppressor gene. Carcinogenic and tumor suppressor miRNAs directly regulate tumor cell proliferation, differentiation, and apoptosis by positively or negatively regulating tumor suppressor genes, oncogenes, or genes that control cell cycle process, differentiation or apoptosis, and participate in tumor formation, development, and even invasion and metastasis. A large number of researches show that miRNAs have characteristic expression profile changes in tumor cells, carcinoma tissues, para-carcinoma tissues, and normal tissues, as well as characteristic expression level changes in hematuria of tumor patients, which provides new ideas for the diagnosis of tumors, and also suggests that miRNAs may become important molecular biological markers for tumor diagnosis.

Because of having the advantages of less trauma, easy acquisition, repeatability, many detection indicators, etc., peripheral blood has been the main specimen source for detecting clinical disease markers. It is found through researches in recent years that endogenous circulating miRNAs exist in peripheral blood, and may become biological markers for many diseases such as tumor, etc. because of having high stability and specificity. Some researchers have suggested that circulating miRNAs mainly exist in exosome and may become a good source for detecting serum miRNAs. Therefore, if the relevant characteristics of exo-miRNAs are combined, and if a corresponding lung cancer diagnosis kit with high specificity can be developed and then can be used in the field of auxiliary differential diagnosis and scientific research of benign and malignant lung nodules, it will greatly promote lung cancer screening research and the transformation of achievements in scientific research, and will play a huge role in the differentiation and diagnosis of benign and malignant lung tumors.

At present, when the existing microRNAs are used as molecular markers for detecting lung cancer, due to the defects of low accuracy and specificity or high detection costs because there is a need to detect using several markers simultaneously, there is no suitable kit suitable for commercial production.

In addition, the PCR platform-based two-step detection system for miRNA mainly includes a miRNA quantitative detection technology combined with probe method and a dye method-based detection technology: 1), the quantitative detection technology combined with probe method includes Stem-loop RT-PCR probe method, key-like method and Ligation detection. These three methods require the use of miRNA-specific probes, have the outstanding advantage of high specificity, and often can distinguish different variants of the same miRNA family. However, there are phenomena of weak combination between miRNA and Stem-loop RT, and mismatch between a stem-loop primer and non-target miRNA; and 2) The quantitative detection technology based on PCR and fluorescent dye method such as SYBR Green includes poly (A) polymerase tailing method, Stem-loop dye method, primer extension method, Multiplexed RT, etc. Most of the technologies using SYBR Green have higher sensitivity, lower costs and lower specificity. Relatively speaking, for the method of adding PolyA tail and stem-loop structure, the paired sequences of miRNAs are extended, and then normal reverse transcription and subsequent PCR detection are conducted. The stem-loop method is only for mature miRNAs, and has relatively high specificity; and the tailing method can detect mature miRNAs and pre-miRNAs, has relatively low specificity and sensitivity, but has simple operation and primer design.

In recent years, isothermal amplification of nucleic acids has flourished, can amplify specific DNA or RNA at a specific temperature, has advantages that the instrument and reaction time are greatly simplified as compared with the traditional PCR technology, and can better meet the requirements for fast and simple detection. A large number of researches show that isothermal amplification is applied to miRNA detection. The isothermal amplification-based one-step detection method for miRNA is summarized and analyzed: 1. the method can be divided into linear amplification of fluorescence signals in reaction and exponential amplification reaction (EXPAR) according to the amplification type. The linear amplification generally refers to collecting fluorescence signals using a fluorescence spectrophotometer after the reaction ends, conducting quantitative analysis based on the destination Flu fluorescence value; and EXPAR refers to exponentially amplifying detection signals using the EXPAR isothermal amplification technology to achieve a standard S-type amplification curve. For the former, the reaction can be conducted on an ordinary PCR instrument and then destination signals can be collected on the fluorescence spectrophotometer, which is more beneficial to the development of POCT products, but generally has poor detection sensitivity and stability. For the latter, the detection results of accurate quantitative detection of the traditional RT-PCR can be achieved on a real-time fluorescent quantitative PCR instrument using POI (similar to Ct value). 2. Isothermal amplification can also be divided into a probe method and a dye method according to different fluorescence materials used, wherein similar to the traditional RT-PCR, the probe method has high detection specificity and low sensitivity in general, while the dye method is on the contrary. 3. DSN (Duplex-Specific Nuclease), as a thermostable nuclease, does not require specific recognition sites and can selectively degrade DNA in double-strand DNA and DNA-RNA hybrids, but has little effect on single-strand DNA/RNA nucleic acid molecules and double-strand RNA molecules, and can distinguish duplexes completely and incompletely matched. A nicking enzyme, also called nicking endonuclease, is a special enzyme of the restriction endonucleases, recognizes specific nicking sites, only niches one strand of double-strand DNA, causing one nick, and conducts site-specific nicking on DNA molecules. In some researches, quick and simple one-step isothermal miRNA detection technologies are designed based on the functions of specific nicking enzymes such as nicking nicking enzyme, duplex-specific nuclease (DSN) or the like. However, the common disadvantages are that the fluorescence background is high, the lower detection limit is not met, and the required reaction time is overlong when the sensitivity is increased by reducing the reaction temperature and other methods. The present invention greatly expands the design idea by combining different types of fluorescence signals, amplification technologies and specific reagents used, greatly shortens the detection time and improves the detection sensitivity and specificity, and meets the requirements of one-step miRNA clinical detection.

### SUMMARY

By combining the relevant characteristics of exosome and miRNA, the present invention evaluates changes in expression profile of lung cancer serum exo-miRNA and lung cancer tissue exo-miRNA and correlation with tumor, and screens out exosome microRNA molecular markers that diagnose and predict lung cancer.

Preferably, the exosome-related microRNA molecule comprises at least one up-regulated exosome microRNA molecule, or at least one down-regulated exosome microRNA molecule, or at least one up-regulated molecule and at least one down-regulated exosome microRNA molecule.

Preferably, the up-regulated exosome microRNA molecule is at least one of miR-21, miR-486-5p, miR-205 and miR-126, and the down-regulated microRNA molecule is at least one of miR-152, Let-7a and miR-148a.

More preferably, in the embodiment of the application of the microRNA molecular marker in diagnosis and prediction of lung cancer, wherein at least one or several microRNAs in the up-regulated group and at least one or several microRNAs in the down-regulated group are combined for detection, and more preferably, the marker is a combination of miR-21 and Let-7a, a combination of miR-205 and Let-7a, a combination of miR-126 and miR-152 or a combination of miR-486-5p and miR-148a.

More preferably, the diagnosis and predication specifically include lung cancer screening, auxiliary diagnosis, therapeutic effect evaluation, prognosis evaluation or relapse monitoring.

In the application of the microRNA molecular marker in the diagnosis and prediction of lung cancer, the molecular marker is derived from body fluid or cell; and the body fluid comprises at least one of blood, sputum, pleural effusion, pleural lavage fluid, urine and saliva.

A detection kit for auxiliary diagnosis of lung cancer, which is a PCR platform-based two-step detection kit for miRNA, wherein all two-step detection systems described in the Description provide the theoretical basis for constructing the kit.The kit includes specific stem-loop RT-primers of microRNA molecular markers, PCR forward primers, PCR universal reverse primers, and specific probes for detecting microRNA molecular markers, wherein the microRNA molecular markers are at least two markers, wherein one is selected from up-regulated markers miR-21, miR-486-5p, miR-205 or miR-126; and the other is selected from the down-regulated markers miR-152, Let-7a or miR-148a.

Preferably, the loop of the neck of the specific stem-loop RT-Primer is designed with a discontinuous complementary base pair TGCG and CGCA to form a key-like structure, and a short arm is connected to the microRNA molecule through a ligase during a reverse transcription reaction.

More preferably, the RT-primer sequence of the molecular marker miR-21 is as shown in SEQ ID NO. 1:

5′-GATGAGGAGTGTCGTGGAGTCGGCAATTTCCTCATCATCAACAT-3′;

the PCR forward primer sequence of the miR-21 is as shown in SEQ ID NO. 2:

5′-CTCCGTCAGGGTAGCTTATCAGACTG-3;

the PCR universal reverse primer sequence of miR-21 is as shown in SEQ ID NO. 3;

5′-CTCAAGTGTCGTGGAGTCGGC-3′;

the specific probe sequence of miR-21 is as shown in SEQ ID NO. 4;

5′-FAM-TTTCCTCATCATCAACAT-MGB-3′

the RT-primer sequence of the molecular marker miR-486-5p is as shown in SEQ ID NO. 5;

5′-GATGAGGAGTGTCGTGGAGTCGGCAATTTCCTCATCACTCGGGG-3′;

the PCR forward primer sequence of miR-486-5p is as shown in SEQ ID NO. 6:

5′-CTCCGTCAGGGTCCTGTACTGAGCTG-3′;

the PCR universal reverse primer sequence of miR-486-5p is as shown in SEQ ID NO. 3:

5′-CTCAAGTGTCGTGGAGTCGGC-3′;

the specific probe sequence of miR-486-5p is as shown in SEQ ID NO. 7:

5′-FAM-TTTCCTCATCACTCGGGG-MGB-3′

the RT-primer sequence of the molecular marker miR-205 is as shown in SEQ ID NO. 8:

5′-GATGAGGAGTGTCGTGGAGTCGGCAATTTCCTCATCACAGACTC-3′;

the PCR forward primer sequence of miR-205 is as shown in SEQ ID NO. 9:

5′-CTCCGTCAGGGTCCTTCATTCCACCG-3′;

the PCR universal reverse primer sequence of miR-205 is as shown in SEQ ID NO. 3:

5′-CTCAAGTGTCGTGGAGTCGGC-3′;

the specific probe sequence of miR-205 is as shown in SEQ ID NO.10:

5′-FAM-TTTCCTCATCACAGACTC-MGB-3′;

the RT-primer sequence of the molecular marker miR-126 is as shown in SEQ ID NO. 11:

5′-GATGAGGAGTGTCGTGGAGTCGGCAATTTCCTCATCACGCATTA-3′:

the PCR forward primer sequence of miR-126 is as shown in SEQ ID NO. 12:

5′-CTCCGTCAGGGTCGTACCGTGAGTAA-3′;

the PCR universal reverse primer sequence of miR-126 is as shown in SEQ ID NO. 3:

5′-CTCAAGTGTCGTGGAGTCGGC-3′;

the specific probe sequence of miR-126 is as shown in SEQ ID NO. 13:

5′-FAM-TTTCCTCATCACGCATTA-MGB-3′

the RT-primer sequence of the molecular marker let-7a is as shown in SEQ ID NO. 14:

5′-GATGAGGAGTGTCGTGGAGTCGGCAATTTCCTCATCAACTATAC-3′;

the PCR forward primer sequence of let-7a is as shown in SEQ ID NO. 15:

5′-CTCCGTCAGGGTGAGGTAGTAGGTT-3′;

the PCR universal reverse primer sequence of let-7a is as shown in SEQ ID NO. 3:

5′-CTCAAGTGTCGTGGAGTCGGC-3′;

the specific probe sequence of let-7a is as shown in SEQ ID NO. 16:

5′-FAM-TTTCCTCATCAACTATAC-MGB-3′

the RT-primer sequence of the molecular marker miR-152 is as shown in SEQ ID NO.17:

5′-GATGAGGAGTGTCGTGGAGTCGGCAATTTCCTCATCAAGTCGGAG-3′;

the PCR forward primer sequence of miR-152 is as shown in SEQ ID NO. 18:

5′-CTCCGTCAGGGAGGTTCTGTGATACA-3′;

the PCR universal reverse primer sequence of miR-152 is as shown in SEQ ID NO. 3:

5′-CTCAAGTGTCGTGGAGTCGGC-3′;

the specific probe sequence of miR-152 is as shown in SEQ ID NO. 19:

5′-FAM-TTTCCTCATCAAGTCGGAG-MGB-3′;

the RT-primer sequence of the molecular marker miR-148a is as shown in SEQ ID NO. 20:

5′-GATGAGGAGTGTCGTGGAGTCGGCAATTTCCTCATCAAGTCGGAG-3′;

the PCR forward primer sequence of miR-148a is as shown in SEQ ID NO. 21:

5′-CTCCGTCAGGGAAAGTTCTGAGACA-3′;

the PCR universal reverse primer sequence of miR-148a is as shown in SEQ ID NO. 3:

5′-CTCAAGTGTCGTGGAGTCGGC-3′;

the specific probe sequence of miR-148a is as shown in SEQ ID NO. 22:

5′-FAM-TTTCCTCATCAAGTCGGAG-MGB-3′;

More preferably, the kit further comprises miRNA molecular marker standards, wherein the miR-21 molecular marker standard is miR-21, diluted to a concentration of 10¹³ copy/μL; the miR-486-5p molecular marker standard is miR-486-5p, diluted to a concentration of 10¹³ copy/μL; the miR-205 molecular marker standard is miR-205, diluted to a concentration of 10¹³ copy/μL; the miR-126 molecular marker standard is miR-126, diluted to a concentration of 10¹³ copy/μL; the let-7a molecular marker standard is let-7a, diluted to a concentration of 10¹³ copy/μL; the miR-152 molecular marker standard is miR-152, diluted to a concentration of 10¹³ copy/μL; and the miR-148a molecular marker standard is miR-148a, diluted to a concentration of 10¹³ copy/μL.

The kit further comprises specific amplification templates of microRNA molecules, Vent (exo-) DNA polymerase, nicking enzyme, duplex-specific nuclease and molecular hybridization probes.

An isothermal amplification-based diagnosis kit for miRNA, wherein the first amplification template sequence of the molecular marker miR-21 is as shown in SEQ ID NO. 23:

5′-GTCATCGCAGACAACCTCATCTAGACTCATCAACATCAGTCTGATAAGCTAA-NH2-3′

the second amplification template sequence of miR-21 is as shown in SEQ ID NO. 24:

5′-ATCAACATCAGTCTGATAAGCTAATCTAGACTCGTCATCGCAGACAACCTCA-NH2-3′

the hybridization probe sequence of miR-21 is as shown in SEQ ID NO. 25:

5′-FAM-AGCCTATCAACATCAGTCTGATAAGCTAATAGGCTGCATC-Tamra-3′

the first amplification template sequence of the molecular marker miR-486-5p is as shown in SEQ ID NO. 26:

5′-GTCATCGCAGTGTTCCTCAACAGACTCTCTCGGGGCAGCTCAGTACAGGAA-NH2-3′

the second amplification template sequence of miR-486-5p is as shown in SEQ ID NO. 27:

5′-CTCGGGGCAGCTCAGTACAGGAAAACAGACTCAGTCATCGCAGTGTTCCTCA-NH2-3′

the hybridization probe sequence of miR-486-5p is as shown in SEQ ID NO. 28:

5′-FAM-AGCCTAACTCGGGGCAGCTCAGTACAGGAATAGGCTGCATC-Tamra-3′

the first amplification template sequence of the molecular marker miR-205 is as shown in SEQ ID NO. 29:

5′-GTCATCGCAGTGTTCCTCAACAGACTCTCAGACTCCGGTGGAATGAAGGAA-NH2-3′

the second amplification template sequence of miR-205 is as shown in SEQ ID NO. 30:

5′-CAGACTCCGGTGGAATGAAGGAAACAGACTCAGTCATCGCAGTGTTCCTCA-NH2-3′

the hybridization probe sequence of miR-205 is as shown in SEQ ID NO. 31:

5′-FAM-AGCCTAACAGACTCCGGTGGAATGAAGGAATAGGCTGCATC-Tamra-3′

the first amplification template sequence of the molecular marker miR-126 is as shown in SEQ ID NO. 32:

5′-GTCATCGCAGTGTTCCTCAACAGACTCTCGCATTATTACTCACGGTACGAA-NH2-3′

the second amplification template sequence of miR-126 is as shown in SEQ ID NO. 33:

5′-CGCATTATTACTCACGGTACGAAACAGACTCAGTCATCGCAGTGTTCCTCA-NH2-3′

the hybridization probe sequence of miR-126 is as shown in SEQ ID NO. 34:

5′-FAM-AGCCTAACGCATTATTACTCACGGTACGAATAGGCTGCATC-Tamra-3′

the first amplification template sequence of the Internal control gene Let-7a is as shown in SEQ ID NO. 35:

5′-GTC ATC GCAGTGTTCCTCAACAGACTCTAACTATACAACCTACTACCTCA-NH2-3′

the second amplification template sequence of Let-7a is as shown in SEQ ID NO. 36:

5′-AACTATACAACCTACTACCTCAAACAGACTCAGTCATCGCAGTGTTCCTCA-NH2-3′

the hybridization probe sequence of Let-7a is as shown in SEQ ID NO. 37:

5′-FAM-AGCCTAAACTATACAACCTACTACCTCAATAGGCTGCATC-Tamra-3′

More preferably, the kit further comprises miRNA molecular marker standards, wherein the miR-21 molecular marker standard is miR-21, diluted to a concentration of 10¹³ copy/μL, and diluted into a gradient standard; the miR-486-5p molecular marker standard ismiR-486-5p, diluted to a concentration of 10¹³ copy/μL, and diluted into a gradient standard; the miR-205 molecular marker standard is miR-205, diluted to a concentration of 10¹³ copy/μL, and diluted into a gradient standard; the miR-126 molecular marker standard is miR-126, diluted to a concentration of 10¹³ copy/μL, and diluted into a gradient standard; and the Let-7a molecular marker standard is Let-7a, diluted to a concentration of 10¹³ copy/μL, and diluted into a gradient standard.

In one embodiment, the level of at least one microRNA gene product from the test sample is higher than that of a corresponding microRNA gene product from the control sample (that is, the expression of the microRNA gene product is “up-regulated”. When the amount of microRNA gene products from a subject sample is higher than that of same gene products from a control sample, the expression of the microRNA gene product is “up-regulated”.

In another embodiment, the level of at least one microRNA gene product from the test sample is lower than that of a corresponding microRNA gene product from the control sample (that is, the expression of the microRNA gene product is “down-regulated”. When the amount of microRNA gene products generated by microRNA genes from a subject sample is lower than that of gene products generated by the same genes from a control sample, the expression of the microRNA gene product is “down-regulated”.

In a preferred embodiment, at least one up-regulated microRNA in the test sample is combined with at least one down-regulated microRNA, thus predicting the risk of disease.

PCR Platform-Based Two-Step Detection Kit for miRNA

RT-Primer: the specific RT primer of the present invention integrates with the design advantages of the Stem-loop RT-PCR method and the key-like method: 1. The neck Stem base pair of the Stem-loop RT primer (FIG. 1) is extended, and four pairs of discontinuous complementary base pairs are designed in the loop region to enhance the capacity for forming a key-like structure, so that the RT primer can better maintain the stem-loop structure during the entire reverse transcription process. Thus, not only mismatch between the Stem-loop RT primer and the target miRNA is eliminated and the specificity is improved, but also the number of bases of the reverse transcription product is increased, thereby being more beneficial to subsequent PCR detection. 2. Stem-loop RT has five pairs of bases that are fully complementary to the miRNA, and an enzyme ligation step is added before reverse transcription (FIG. 1), so that the miRNA is combined with the Stem-loop RT firmly and the efficiency of reverse transcription is enhanced. 3. The present invention can be used for conducting PCR detection on the miRNA RT products using the Stem-loop RT primers, and can also be used for conducting PCR detection using fluorescent dye method.

PCR forward and reverse primers: the specific forward primer is added with a Tag so that the amplification template is extended and the amplification efficiency is increased, and the reverse primer is adjusted to enable the Tm values of the forward and reverse primers to be substantially the same, so that the forward and reverse primers can be simultaneously combined with the template and amplified after PCR pre-denaturation, and annealing and extension are conducted at the same temperature.

Hydrolysis probe: the present invention designs a specific hydrolysis probe (FIG. 1) complementary to the template using a design method of TaqMan technology to enhance the specificity of detection.

Quantitative detection is conducted on a miRNA marker, the miRNA is selected as the internal control gene of the miRNA marker, and according to the CP value, the multiple change in the relative expression quantity of the marker is calculated by using a relative quantification formula (2^{-ΔΔCp}) to calculate the score of the miRNA; and the correlation between the relative expression quantity of the miRNA marker and patients with demographic characteristics, benign lesions, and healthy individuals is analyzed using the Pearson correlation coefficient. The clinical pathological diagnosis is used as a reference standard to decide the sensibility and specificity of the miRNA marker. The clinical pathological diagnosis is used as a reference standard to determine the sensibility and specificity of the miRNA marker. The accuracy of the combined detection of the miRNA is determined using the ROC characteristic curve and AUC analysis, and the sample results are interpreted with cut off values.

Combined detection is conducted on two or more miRNA markers, to select 1) the up-regulation of at least one of miR-21, miR-486-5p, miR-205 or miR-126; 2) the down-regulation of at least one of miR-152, Let-7a and miR-148a; and 3) the combined use of the up-regulated molecular marker and the down-regulated molecular marker. According to the CP value, the relative expression quantity 2^{-ΔΔCp} is calculated using a relative quantitative formula to calculate the score of each miRNA. The correlation between the score of the relative expression quantity of each miRNA marker and patients with demographic characteristics, benign lesions, and healthy individuals is analyze using the Pearson correlation coefficient. The clinical pathological diagnosis is used as a reference standard to decide the sensibility and specificity of each miRNA marker. The clinical pathological diagnosis is used as a reference standard to determine the sensibility and specificity of the miRNA marker. A binary logistic regression equation is obtained using logistic regression models, and a best diagnosis combination of the miRNA markers is selected. The accuracy of the combined detection of the miRNA is determined using the ROC characteristic curve and AUC analysis, and the sample results are interpreted with cut off values.

Isothermal Amplification-Based One-Step Detection System for miRNA

### One-Step Detection System of EXPAR

Amplification template: the specific amplification templates A and B of the present invention are divided into 3 parts (FIG. 2), wherein the first part is completely complementary to the miRNA, which is beneficial to detecting the specificity; the second part is a recognition nicking site of a corresponding nicking enzyme, at which newly single strands (tirggers) are circularly nicked, displaced and released under the action of specific polymerase; and the third part is a tirggers complementary strand, which can continuously release tirggers. Once the miRNA is combined with the amplification template A, it will trigger an EXPAR 1 reaction, after tirggers as products are continuously released, it will be combined with the amplification template B to trigger an EXPAR 2 reaction, and after New tirggers as products are continuously released, it will return to combine with the template A to enter the EXPAR 1 reaction. Characteristics: 1. The sequence of the third part can be basically designed at random to prevent an amplification template from forming a secondary structure and then reducing the fluorescence background; 2. In the amplification templates A and B, only the first part and the third part are reversed, and thus no primer dimer is formed between A and B; 3. Two cycles of amplification are isothermally achieved using the one-step method, one cycle strand reaction is formed by connecting two continuous SDA reactions in series, thereby achieving the EXPAR cycle mode, and completing an amplification reaction within 30 minutes.

### One-Step Detection System of Isothermal Linear Amplification

DSN (Duplex-Specific Nuclease can selectively degrade DNA strands in double-strand DNA and DNA-RNA hybrids, but has little effect on RNA strands in single-strand DNA/RNA nucleic acid molecules and double-strand RNA molecules. The molecular beacon (MB) probe designed by the present invention based on the linear amplification technology of isothermal signals specifically hybridizes with the miRNA, degrades the DNA probe strand of the DNA-RNA hybrid double strands and releases fluorescence signals under the action of the DSN enzyme, and the miRNA hybridizes with the probe again to enter the next cycle, thereby achieving the purpose of fluorescence signal amplification. Destination signals can be collected using an ordinary fluorescence spectrophotometer, which is beneficial to the development of POCT products (FIG. 3). Characteristics: 1. Using the molecular beacon probe, the loop is completely complementary to the miRNA, and the specificity is high so that single bases can be distinguished; 2. Five pairs of bases on the neck are complementary, which can ensure that the probe maintains the stem-loop structure in the free state, reducing fluorescence background signals, and can quickly form a rigid chain-like template when hybridizing with the miRNA, improving combination efficiency; 3. There is no PCR target product amplification, having less pollution; 4. The DSN enzyme does not require specific recognition sites, which can be applied to all miRNA detection; and5. The operation is simple, the reagent consumable is less, and the cost is low.

The Present Invention has the Following Advantageous Effects that:

(1) An application of microRNA molecular markers in auxiliary diagnosis reagents for lung cancer, including: 1) the up-regulation of at least one of miR-21, miR-486-5p, miR-205 or miR-126; 2) the down-regulation of at least one of miR-152, Let-7a or miR-148a; and 3) the combined use of the up-regulated molecular marker and the down-regulated molecular marker. Through big-data clinical verification experiments, it has outstanding advantage in the aspect of auxiliary diagnosis indicators of lung cancer, which greatly improves the accuracy of relative quantification when conducting miRNA detection.

(2) The present invention optimizes and improves the existing detection methods, and develops a PCR platform-based two-step detection kit for miRNA, so that the corresponding detection and analysis methods can be selected according to the purpose of the detection and the requirements of experimental conditions. Due to the effectiveness of miRNA and the correlation with the tumor of lung cancer, the kit can be used for distinguishing benign and malignant lung nodules in the early stage, and can also be used for monitoring prognosis, pre-operation postoperation, treatment, and therapeutic effect in real time. The two-step detection system includes independently designed specific stem-loop RT primers, PCR forward and reverse primers, and Taqman probe primers, wherein the specific primers enable the miRNA detection specificity to distinguish single base differences and the sensitivity to reach the lower detection limit of 1 copy/μL to a minimum extent, greatly improving the detection efficiency and accuracy of miRNA. In addition, the PCR thermal cycle conditions of different markers are the same, so that not only multiple markers can be detectioned in the same batch and the same template, improving the detection accuracy and detection efficiency of combined markers, but also time and costs can be reduced.

(3) The isothermal amplification-based one-step detection system for miRNA developed by the present invention is faster and more convenient, which can greatly improve detection efficiency and reduce detection costs.

(4) The serum exosome exo-miRNA is adopted as a marker for the combined detection, and the effect is better compared with that produced by direct detection ofthe plasma exosome-miRNA, serum-miRNA or plasma-miRNA. The exo-miRNA has good stability, the exo-miRNA can still be extracted effectively after the serum is stored at 4° C. for 20 days, and the extracted exo-miRNA can be cryopreserved at −20° C. for 50 days, and can be preserved at −80° C. for long time.

### BRIEF DESCRIPTION OF THE DRAWINGS

FIG. 1 is a schematic diagram of two-step PCR detection amplification for miRNA.

FIG. 2 is a schematic diagram of one-step EXPAR.

FIG. 3 is a schematic diagram of one-step isothermal linear amplification.

FIG. 4 is a PCR standard curve and detection sensitivity of a miRNA marker (A: PCR minimum lower detection limit of Let-7a; B: PCR standard curve of Let-7a; C: PCR minimum lower detection limit of miR-21; D: PCR standard curve of miR-21; E: PCR minimum lower detection limit of miR-486-5p; F: PCR standard curve of miR-486-5p; G: PCR minimum lower detection limit of miR-205; H: PCR standard curve of miR-205; I: PCR minimum lower detection limit of miR-126; J: PCR standard curve of miR-126; K: PCR minimum lower detection limit of miR-152; L: PCR standard curve of miR-152; M: PCR minimum lower detection limit of miR-148a; N: PCR standard curve of miR-148a).

FIG. 5 is detection stability results for clinical samples through two-step miRNA detection system (A: CV difference value within a batch; B: CV difference value among batches).

FIG. 6 is detection results of combination of miR-21 and Let-7a in a clinical sample of lung cancer (A: detection results of combination of miR-21 and Let-7a in a tissue sample of lung cancer, A-1: miRNA detection of combination of miR-21 and Let-7a; A-2: ROC curve of combination of miR-21 and Let-7a; B: detection results of combination of miR-21 and Let-7a in serum exosome of lung cancer; B-1: relative quantitative results of miRNA detection by combination of miR-21 and Let-7a in serum exosome of lung cancer; B-2: ROC curve of detection by combination of miR-21 and Let-7a in serum exosome of lung cancer; B-3: logistic regression analysis results of detection by combination of miR-21 and Let-7a in serum exosome of lung cancer; B-4: logistic regression ROC curve of detection by combination of miR-21 and Let-7a in serum exosome of lung cancer; B-5: miRNA detection results of combination of miR-21 and Let-7a in plasma exosome of lung cancer; B-6: ROC curve of detection by combination of miR-21 and Let-7a in plasma exosome of lung cancer; B-7: miRNA detection by combination of miR-21 and Let-7a in urine exosome of lung cancer; B-8: ROC curve of detection by combination of miR-21 and Let-7a in urine exosome of lung cancer).

FIG. 7 is detection results of combination of miR-205 and Let-7a in a clinical sample of lung cancer (A: detection miRNA results of combination of miR-205 and Let-7a in a tissue sample of lung cancer; B: ROC curve of detection by combination of miR-205 and Let-7a in a tissue sample of lung cancer; C: detection miRNA results of combination of miR-205 and Let-7a in serum exosome of lung cancer; D: ROC curve of combination of miR-205 and Let-7a in serum exosome of lung cancer; E: detection miRNA results of combination of miR-205 and Let-7a in plasma exosome of lung cancer; F: ROC curve of detection by combination of miR-205 and Let-7a in plasma exosome of lung cancer; G: detection miRNA results of combination of miR-205 and Let-7a in urine exosome of lung cancer; H: ROC curve of detection by combination of miR-205 and Let-7a in urine exosome of lung cancer).

FIG. 8 is detection results of combination of miR-126 and miR-152 in a clinical sample of lung cancer (A: detection results of combination of miR-126 and miR-152 in a tissue sample of lung cancer; B: ROC curve of detection by combination of miR-126 and miR-152 in a tissue sample of lung cancer; C: detection results of combination of miR-126 and miR-152 in serum exosome of lung cancer; D: ROC curve of detection by combination of miR-126 and miR-152 in serum exosome of lung cancer; E: detection results of combination of miR-126 and miR-152 in plasma exosome of lung cancer; F: ROC curve of detection by combination of miR-126 and miR-152 in plasma exosome of lung cancer; G: detection results of combination of miR-126 and miR-152 in urine exosome of lung cancer; H: ROC curve of detection by combination of miR-126 and miR-152 in urine exosome of lung cancer).

FIG. 9 is detection results of combination of miR-486-5p and miR-148a in a clinical sample of lung cancer (A: detection results of combination of miR-486-5p and miR-148a in a tissue sample of lung cancer; B: ROC curve of detection by combination of miR-486-5p and miR-148a in a tissue sample of lung cancer; C: detection results of combination of miR-486-5p and miR-148a in serum exosome of lung cancer; D: ROC curve of detection by combination of miR-486-5p and miR-148a in serum exosome of lung cancer; E: detection results of combination of miR-486-5p and miR-148a in plasma exosome of lung cancer; F: ROC curve of detection by combination of miR-486-5p and miR-148a in plasma exosome of lung cancer; G: detection results of combination of miR-486-5p and miR-148a in urine exosome of lung cancer; H: detection ROC curve of combination of miR-486-5p and miR-148a in urine exosome of lung cancer).

FIG. 10 is expression levels and differences thereof of miRNA in exosome before and after surgery (A: expression levels and differences thereof in serum exosome before and after surgery (A-1: miR-21 detection results, A-2: miRNA-205 detection results, A-3: miRNA-126 detection results, A-4: miRNA-486-5p detection results, A-5: Let-7a detection results, A-6: miR-152 detection results and A-7: miR-148a detection results); B: expression levels and differences thereof in plasma exosome before and after surgery (B-1: miR-21 detection results, B-2: miRNA-205 detection results, B-3: miRNA-126 detection results, B-4: miRNA-486-5p detection results, B-5: Let-7a detection results, B-6: miR-152 detection results and B-7: miR-148a detection results); C: expression levels and differences thereof in urine exosome before and after surgery (C-1: miR-21 detection results, C-2: miRNA-205 detection results, C-3: miRNA-126 detection results, C-4: miRNA-486-5p detection results, C-5: Let-7a detection results, C-6: miR-152 detection results and C-7: miR-148a detection results)).

FIG. 11 is prognosis evaluation for lung cancer through detection by combination of miR-21 and Let-7a in exosome (A: prognosis evaluation for lung cancer through detection by combination of miR-21 and Let-7a in serum exosome, A-1: expression levels of miR-21 and Let-7a, A-2: ROC curve; A-3: progression-free survival curve; A-4: overall survival; B: prognosis evaluation for lung cancer through detection by combination of miR-21 and Let-7a in plasma exosome, B-1: expression levels of miR-21 and Let-7a, B-2: ROC curve; B-3: progression-free survival curve; B-4: overall survival; C: prognosis evaluation for lung cancer through detection by combination of miR-21 and Let-7a in urine exosome; C-1: expression levels of miR-21 and Let-7a, C: ROC curve; C-3: overall survival).

FIG. 12 is prognosis evaluation for lung cancer through detection by combination of miR-205 and Let-7a in exosome (A: prognosis evaluation for lung cancer through detection by combination of miR-205 and Let-7a in serum exosome , A-1: expression levels of miR-205 and Let-7a, A-2: ROC curve; A-3: progression-free survival curve; A-4: overall survival; B: prognosis evaluation for lung cancer through detection by combination of miR-205 and Let-7a in plasma exosome, B-1: expression levels of miR-205 and Let-7a, B-2: ROC curve; B-3: progression-free survival curve; B-4: overall survival; C: prognosis evaluation for lung cancer through detection of combination of miR-205 and Let-7a in urine exosome; C-1: expression levels of miR-205 and Let-7a, C: ROC curve; C-3: overall survival).

FIG. 13 is prognosis evaluation for lung cancer through miR-126 and miR-152 in exosome (A: prognosis evaluation for lung cancer through detection of combination of miR-126 and miR-152 in serum exosome, A-1: expression levels of miR-126 and miR-152, A-2: ROC curve; A-3: overall survival; B: prognosis evaluation for lung cancer through detection of combination of miR-126 and miR-152 in plasma exosome, B-1: expression levels of miR-126 and miR-152, B-2: ROC curve; B-3: overall survival; C: prognosis evaluation for lung cancer through detection by combination of miR-126 and miR-152 in urine exosome; C-1: expression levels of miR-126 and miR-152, C: ROC curve; C-3: overall survival).

FIG. 14 is an isothermal amplification-based one-step detection kit for miRNA and application therefor (A: detection sensitivity and standard curve with Let-7a EXPAR one-step method; B: detection of clinical samples with an EXPAR one-step method; C: Let-7a standard curve with an isothermal linear amplification one-step method; D: miR-21 standard curve with an isothermal linear amplification one-step method; E-1: detection results of combination of miR-21 and Let-7a in urine exosome of lung cancer; E-2: ROC curve of detection by combination of miR-21 and Let-7a in urine exosome of lung cancer; F-1: detection results of combination of miR-21 and Let-7a in serum exosome of lung cancer; F-2: ROC curve of detection by combination of miR-21 and Let-7a in serum exosome of lung cancer).

### DETAILED DESCRIPTION

Preferred embodiments of the present invention will be described below in detail in combination with drawings. Experimental methods in which specific conditions are not specified in the preferred embodiment are carried out, usually under conventional conditions or as recommended by the manufacturer.

The technical solution in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1 PCR platform-Based Two-Step Detection System Kit for miRNA

1) The Instruments Used in the Embodiment are as Follows:

4° C. refrigerated centrifuge (Thermo Fisher Fresco17), LightCycler 480 real-time fluorescent quantitative PCR (Roche), clean bench (SW-CJ-1D, Longyang Scientific Instrument Co., Ltd.), conventional PCR (A100, Hangzhou LongGene Scientific Instrument Co., Ltd.).

2) RNA Reverse Transcription Reaction System:

Reagents: the reagents for preparing reverse transcription reaction system comprise RT-Primer (synthesized by Shanghai Invitrogen), miRNA standard powder (synthesized by Shanghai Invitrogen), T4 DNA Ligase (supplier: NEB, Art. No.: M0202S, containing 10×T4 DNA Ligase Buffer, RNase inhibitor (supplier: Fermentas, Art. No.: K1622), Transcriptase (supplier: Shanghai Invitrogen Biotechnology Co., Ltd., Art. No.: K1622, containing RNase inhibitor, dNTPs and nuclease-free water), T4 Polynucleotide Kinase (supplier: NEB, Art. No.: M0201S) and nuclease-free water(supplier: Shanghai Invitrogen Biotechnology Co., Ltd., Art. No.: K1622). The reagents for preparing the reverse transcription reaction system are packaged bottle by bottle, and when used, the reverse transcription system is made in a certain proportion. The reverse transcription reaction system is 20 μL/time, and the subpacked volume is 50 times of consumption, as shown in Table 1.

TABLE 1
General Table of Common Reverse Transcription Reaction System

| Component | Final concentration | | Volume/μL |
|---|---|---|---|
| RT-Primer | 5 | μmol/L | 5 |
| T4 DNALigaseBuffer | 10× | | 2 |
| dNTPs( containing dUTP) | 1 | mmol/L | 2 |
| RNase inhibitor | 20U | μL⁻¹ | 1 |
| Transcriptase | 200U | μL⁻¹ | 1 |
| T4 DNA Ligase | 10U | μL⁻¹ | 0.5 |
| T4 Polynucleotide Kinase | 2.5U | μL⁻¹ | 0.25 |
| Template | | | 5 |
| Nuclease-free water | | | Watering to 20 μL |

The reverse transcription is conducted according to the conditions in Table 2.

TABLE 2
General RNA Reverse Transcription Conditions

| | | Reverse | | |
|---|---|---|---|---|
| Precooling | Connection | transcription | Inactivation | Cooling |
| On ice | 16° C. | 42° C. | 85° C. | 4° C. |
| 5 min | 30 min | 30 min | 5 min | 30 sec |

cDNA is preserved at 4° C. after 10 times of dilution for subsequent PCR amplification.

3) PCR Reaction System:

Reagents: the reagents for preparing PCR reaction system comprise dNTPs (dCTP, dGTP, dATP, dTTP and dUTP(supplier: Thermo Scientific), F primer solution (F primer, synthesized by Shanghai Invitrogen), universal R primer solution (R primer, synthesized by Shanghai Invitrogen), Probe (synthesized by ABI), DNA polymerase (HS Taq, supplier: Takara company, Art. No.: R007A), uracil-DNA glycosylase (UDG, supplier: NEB, Art. No.: M0280S) and pure water (H₂O).

The reagents for preparing PCR reaction system are packaged bottle by bottle, and when used, the PCR reaction system is made in a certain proportion. The PCR reaction system is 20 μL/time, and the subpacked volume is 50 times of consumption, as shown in Table 3.

TABLE 3
Optimized PCR Reaction System

| | | |
|---|---|---|
| Component | Final concentration | Volume(μL) |

| | | |
|---|---|---|
| 10× Buffer | 10× | 2 |

| | | | |
|---|---|---|---|
| MgCl2 | 25 | mmol/L | 3 |
| dNTPs | 1 | mmol/L | 0.8 |
| F | 0.5 | μmol/L | 0.5 |
| R | 0.5 | μmol/L | 0.5 |
| Probe | 0.2 | mol/L | 0.2 |
| HS Tag | 1U | μL⁻¹ | 0.2 |
| UDG | 0.5U | μL⁻¹ | 0.1 |
| Template | | | 2 |
| ddH₂O | | | Watering to 20 μL |

Then, the amplification reaction is conducted according to the conditions in Table 4.

TABLE 4
PCR Thermal Cycling Conditions

| UDG enzyme | | | |
|---|---|---|---|
| decontamination | Predegeneration | Amplification | Cooling |
| 37° C. | 94° C. | 94° C., 15 sec | 50° C., 30 sec |
| 5 min | 5 min | 60° C., 60 sec | |
| | | 50 cycles | |

| | |
|---|---|
| Fluorescent setting | Fluorescent detection channel: FAM; |
| instructions | Fluorescence detecting position: a terminal |
| | at 60° C. in an amplification cycle. |

4) Standard Configuration of a miRNA Two-Step Molecule Marker:

The cDNA stock solution thereof is 10¹² copy/μL after the reverse transcription is conducted on a standard miRNA, 10 μL of cDNA stock solution is taken and diluted to 10¹¹ copy/μL with the addition of 900_, of sterile purified water, and then 10pL 10¹¹ copy/μL of diluent is taken again and diluted to 10¹⁰ copy/μL with the addition of 90 μL of sterile purified water, and is dilute to diluent of 1 copy/μL step by step in sequence.

5) Sensitivity of miRNA Two-Step Detection System:

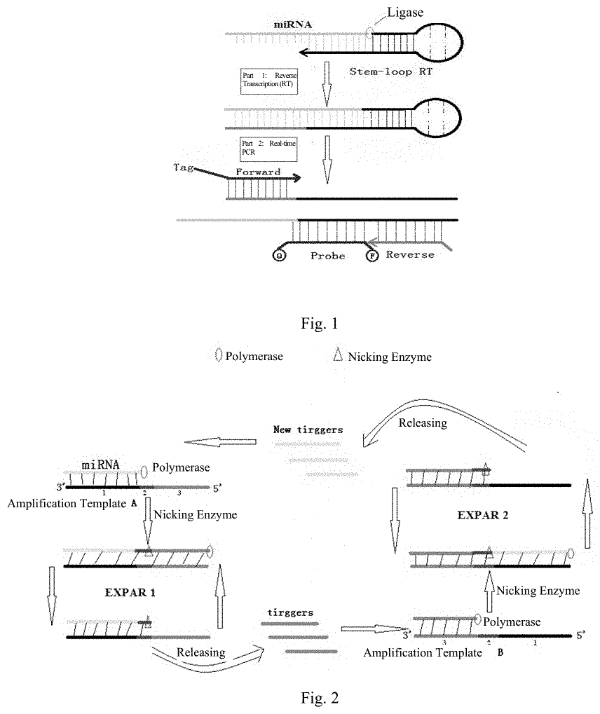

A PCR platform-based two-step detection system kit for miRNA is adopted, and the standard of miR-152, Let-7a, miR-148a, miR-21, miR-486-5p, miR-205 or miR-126 is detected, to obtain lower detection limit and amplification efficiency. A detection principle is shown in FIG. 1.

Taking miR-21 as an example, the standard configuration of a miR-21 two-step molecule marker is as follows:

The cDNA stock solution thereof is 10¹² copy/μL after the reverse transcription is conducted on a standard miR-21, 10 μL of cDNA stock solution is taken and diluted to 10¹¹ copy/μL with the addition of 900_, of sterile purified water, and then 10 μL 10¹¹ copy/μL of diluent is taken again and diluted to 10¹⁰ copy/μL with the addition of 90 μL of sterile purified water, and is dilute to diluent of 1 copy/μL step by step in sequence.

The construction of two-step detection systems of other miRNA molecule markers and the standard configuration refer to miR-21, only the templates, the primers and the probes are different, and the PCR reaction conditions are the same.

The miRNA standard detection results of a two-step detection system are shown in Table 5.

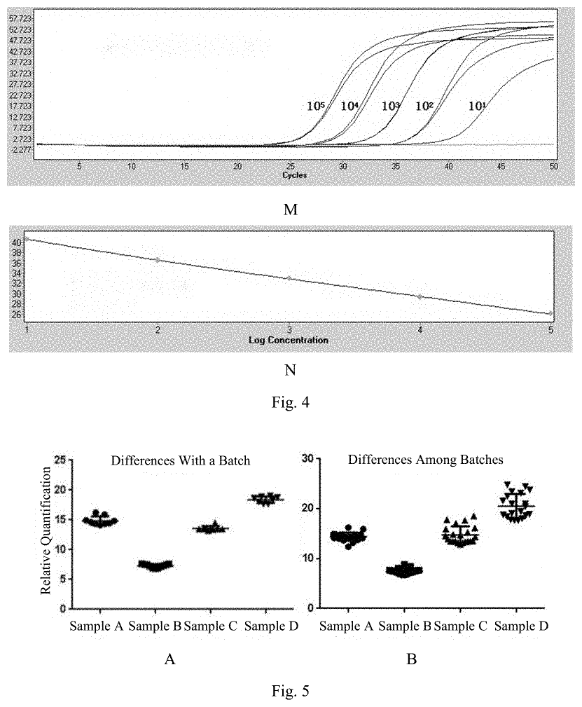

TABLE 5
miRNA Standard detection results

| | Lower | | | |
|---|---|---|---|---|
| | detection | | Correlation | |
| | limit | Amplification | coefficient | |
| Markers | (copy/μL) | efficiency | (R²) | Remarks |
| Let-7a | 1 | 96.3% | >0.99 | FIG. 4, A and B |
| miR-21 | 1 | 101.1% | >0.99 | FIG. 4, C and D |
| miR-486- | 1 | 98.6% | >0.99 | FIG. 4, E and F |
| 5p | | | | |
| miR-205 | 1 | 94.8% | >0.99 | FIG. 4, G and H |
| miR-126 | 1 | 103.2% | >0.99 | FIG. 4, I and J |
| miR-152 | 10 | 101.3% | >0.99 | FIG. 4, K and L |
| miR-148a | 10 | 96.5% | >0.99 | FIG. 4, M and N |

6) Detection Stability Evaluation of a Two-Step miRNA Detection System for Clinical Samples

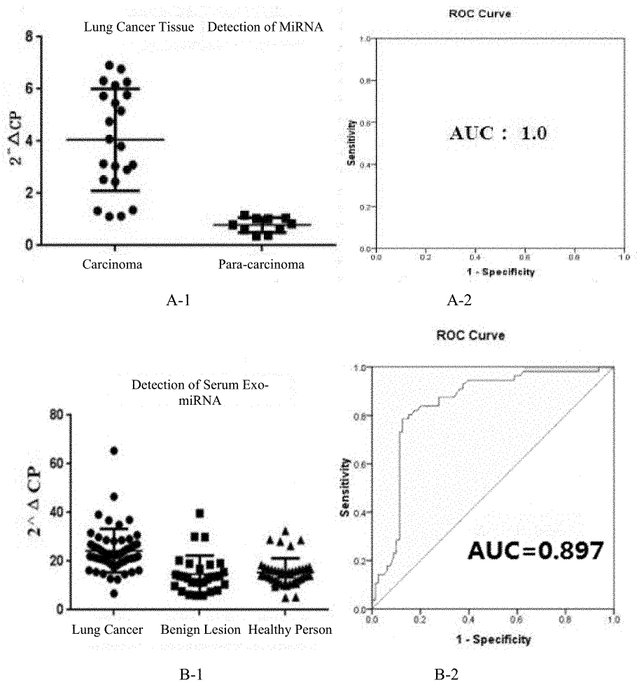

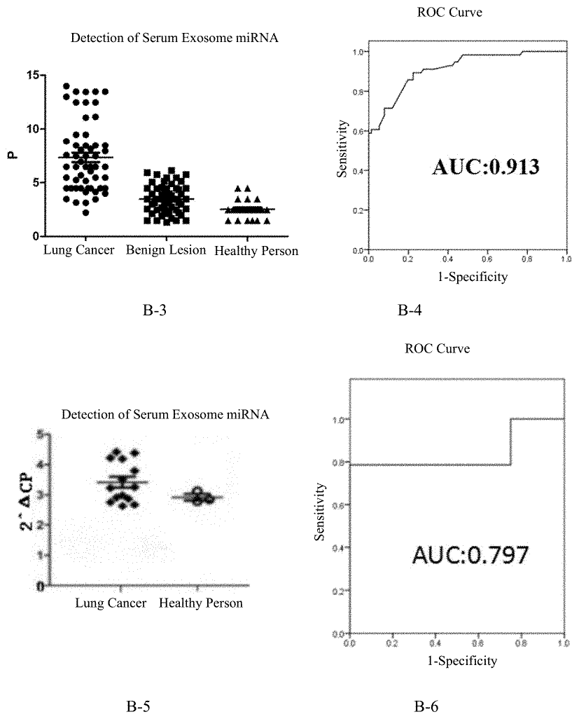

The stability of the detection results is evaluated by combining serum Exo-miR-21 with the down-regulated marker Exo-let-7a for miRNA in clinical samples of lung cancer. There are 4 different clinical serum samples. Each sample is detected in 3 batches. Each batch is duplicated for 3 times, to verify the stability of the detection evaluation system (comprising extraction and purification of Exo-miRNA, reverse transcription and PCR operation detection). The results that the CV difference value of the same sample within a batch can reach within 4%, and the CV difference value among batches can reach within 8%, showing that the miRNA two-step detection evaluation system has favorable stability are shown in FIG. 5.

7) Early Diagnosis of Lung Cancer with a Two-Step Detection Kit for miRNA and Evaluation of Differentiation Effect of Benign and Malignant Pulmonary Nodules

(1) Sample Collection

The tissue, serum, plasma and urine samples from persons with lung cancer diagnosed by hospital examination (comprising different stages, subtypes, gender and age groups), persons with pulmonary benign lesion and healthy persons are collected.

(2) Extraction and Purification of miRNA in Tissue

The miRNAs in the tissue and serum are extracted and purified by adopting a commercial product miRNeasy Serum/Plasma Kit in a QIAGEN company (Art. No. 217184), and by using a Nano-Drop 2000, the RNA nucleic acid mass is measured, the RNA concentration and the purity are recorded, and normalization processing is conducted on the tissue miRNA.

(3) Extraction and Purification of Serum Exosome miRNA and Plasma Exosome miRNA

The serum and plasma exosomes are extracted by adopting a commercial product ExoQuickTM kit in a SBI company (Art.No. EXOQSA-1).The miRNA in the exosome is extracted and purified by adopting the commercial product miRNeasy mini kit in the QIAGEN company (Art.No. 217004), and by using the Nano-Drop 2000, the RNA nucleic acid mass is measured, and the RNA concentration and the purity are recorded.

(4) Extraction and Purification of Urine Exosome miRNA

The urine exosome is extracted by adopting a commercial product ExoQuick-TC for Tissue Culture Media and Urine kit in the SBI company (Art.No. EXOTC10A-1). The miRNA in the exosome is extracted and purified by adopting the commercial product miRNeasy mini kit in the QIAGEN company (Art.No. 217004) , and by using the Nano-Drop 2000, the RNA nucleic acid mass is measured, and the RNA concentration and the purity are recorded.

(5) Two-Step Detection System for miRNA

A PCR platform-based two-step detection system kit for miRNA in embodiment 1 is adopted. The Exo-miRNAs in serum samples from 56 patients with lung cancer in an early stage of Ia stage are detected in control group of 76 patients (healthy persons and persons with benign lesion), to detect expression quantity CP values of miR-152, Let-7a, miR-148a, miR-21, miR-486-5p, miR-205 or miR-126, and according to the CP values, the relative expression quantity is calculated by using a relative quantitative formula.

(6) miRNA Detection Results of Lung-Related Disease

(1) Detection Results of Combination of miR-21 and Let-7a in a Clinical Sample of Lung Cancer

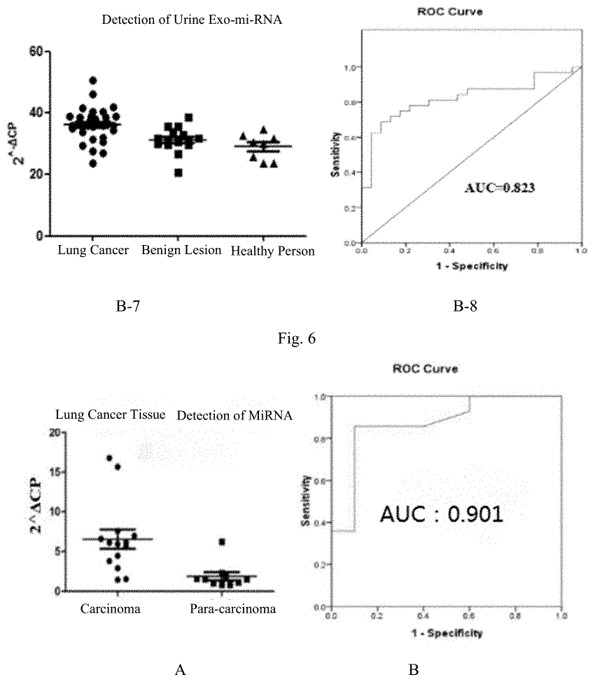

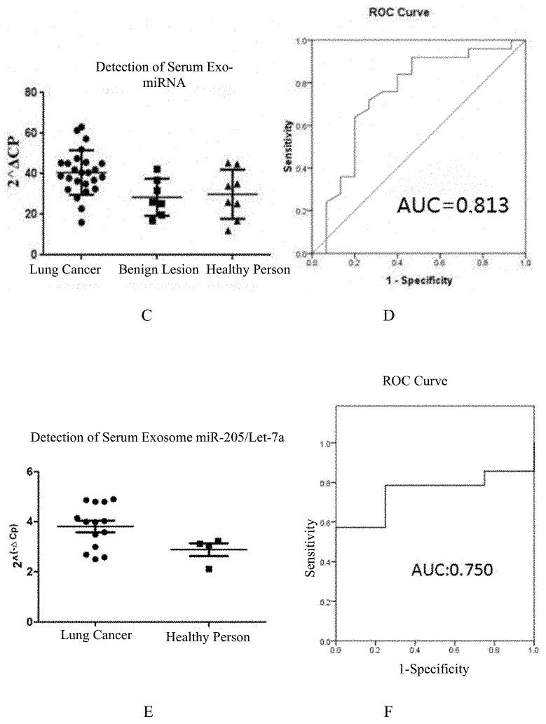

As shown in A of FIG. 6, the miRNAs in carcinoma and para-carcinoma tissue samples from 22 patients with lung cancer are detected, to detect the expression quantity CP values of the up-regulated miR-21 for miRNA and the down-regulated Let-7a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer, with AUC=1.0, showing a significant advantage.

As shown in B in FIG. 6, the Exo-miRNAs in serum samples from 56 patients with lung cancer in an early stage of Ia stage are detected in control group of 76 patients (healthy persons and persons with benign lesion), to detect the expression quantity CP values of the up-regulated marker miR-21 for miRNA and the down-regulated marker Let-7a for miRNA, and according to the CP values multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer, with AUC=0.897, and when Cutoff value is taken as 20.42, the diagnose sensitivity is 82.2% and the specificity is 92.7%, showing a significant advantage (as shown in B-1 and B-2 of FIG. 6). The logistic regression analysis is conducted on the CP(CT) value and copy number obtained by combined diagnosis of markers, to establish a diagnostic model: a P value is calculated through the following formula:

P=EXP(PI)/(1+EXP(PI)), wherein the PI is calculated as follows: PI=50.979+3.462×log[copy(miR-21)]−7.516×log[copy(let-7a)]−1.09×CP(let-7a). With AUC=0.913, when Cutoff value is taken as 0.2063, the diagnose sensitivity is 91% and the specificity is 92%, showing a significant advantage (as shown in B-3 and B-4 of FIG. 6).

As shown in B-5 and B-6 of FIG. 6, the up-regulated marker miR-21 for miRNA and the down-regulated marker Let-7a for miRNA correspond to plasma exosome microRNA in clinical sample in control group of samples from 16 patients with lung cancer in Ia stage and 3 healthy persons. The results show that plasma exosome microRNA has a certain degree of differentiation in control group of lung cancer and healthy persons.

As shown in B-7 and B-8 of FIG. 6, the Exo-miRNAs in urine samples from 32 patients with lung cancer in an early stage of Ia stage are detected in control group of 22 patients (healthy persons and persons with benign lesion), to detect the expression quantity CP values of the up-regulated marker miR-21 for miRNA and the down-regulated marker Let-7a for miRNA, thus obtaining the score of the relative expression quantity for miRNA. The detection results are analyzed through t detection by using SPSS17.0, to obtain P<0.05, indicating that the combined markers are significantly correlated with the prediction of early lung cancer, with AUC=0.823, and when Cutoff value is taken as 23.6645, the diagnose sensitivity is 87.5% and the specificity is 78.3%, showing a significant advantage.

Compared with the effect of tumor tissue needle biopsy and the diagnostic effect of plasma exosome miRNA, the plasma exosome miRNA has the significant advantages of noninvasive diagnostic effect by showing diagnostic effects of tissue, serum and plasma exosome miRNA markers.

(2) Detection Results of Combination of miR-205 and Let-7a in a Clinical Sample of Lung Cancer

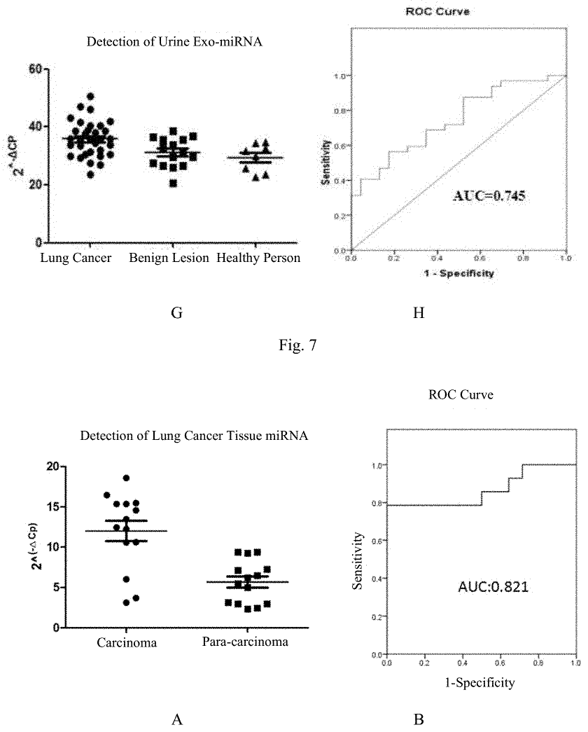

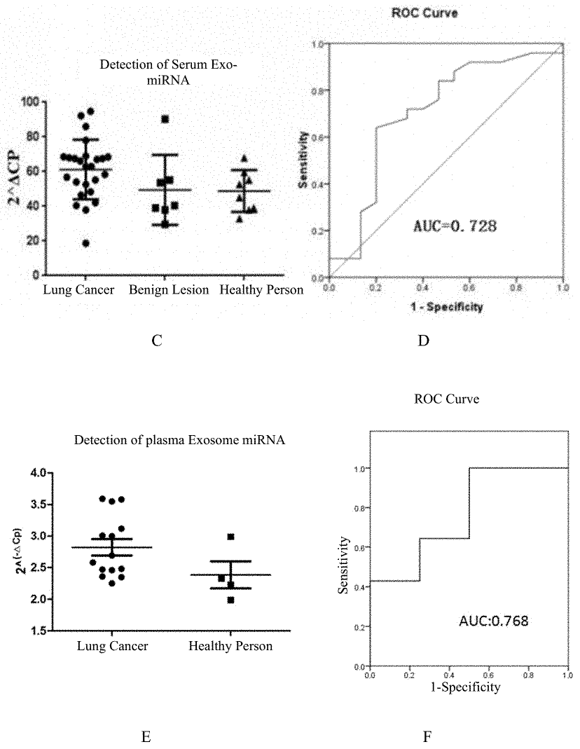

As shown in B of FIG. 7, the miRNAs in carcinoma and para-carcinoma tissue samples from 14 patients with lung cancer are detected, to detect the expression quantity CP values of the up-regulated marker miR-205 for miRNA and the down-regulated marker Let-7a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.901, showing a significant advantage.

As shown in C and D in FIG. 7, the Exo-miRNAs in serum samples from 25 patients with lung cancer in an early stage of Ia stage are detected in control group of 15 patients (healthy persons and persons with benign lesion), to detect the expression quantity CP values of the up-regulated marker miR-205 for miRNA and the down-regulated marker Let-7a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.803, and when Cutoff value is taken as 37.32, the diagnose sensitivity is 76.1% and the specificity is 83.0%, showing a significant advantage.

As shown in E and F of FIG. 7, the up-regulated marker miR-205 for miRNA and the down-regulated marker Let-7a for miRNA correspond to plasma exosome microRNA in clinical sample in control group of samples from 16 patients with lung cancer in Ia stage and 3 healthy persons. The results show that plasma exosome microRNA has a certain degree of differentiation in control group of lung cancer and healthy persons.

As shown in G and H of FIG. 7, the Exo-miRNAs in urine samples from 25 patients with lung cancer in an early stage of Ia stage are detected in control group of 15 patients (healthy persons and persons with benign lesion), to detect the expression quantity CP values of up-regulated marker miR-21 for miRNA and down-regulated marker Let-7a for miRNA, and according to the CP values, the score of the relative expression quantity for miRNA is obtained. The detection results are analyzed through t detection by using SPSS17.0, to obtain P<0.05, indicating that the combined markers are significantly correlated with the prediction of early lung cancer, with AUC=0.745, and when Cutoff value is taken as 29.348, the diagnose sensitivity is 83.5% and the specificity is 65.2%, showing a significant advantage.

(3) Detection Results of Combination of miR-126 and miR-152 in a Clinical Sample of Lung Cancer

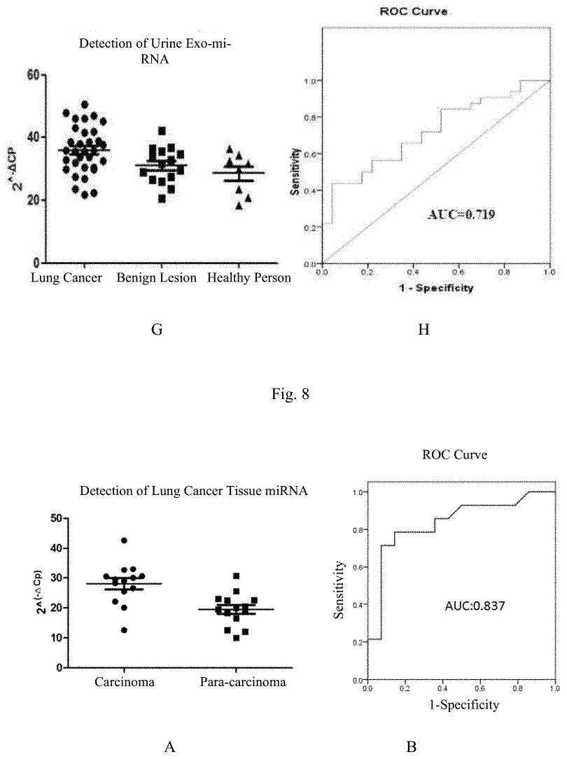

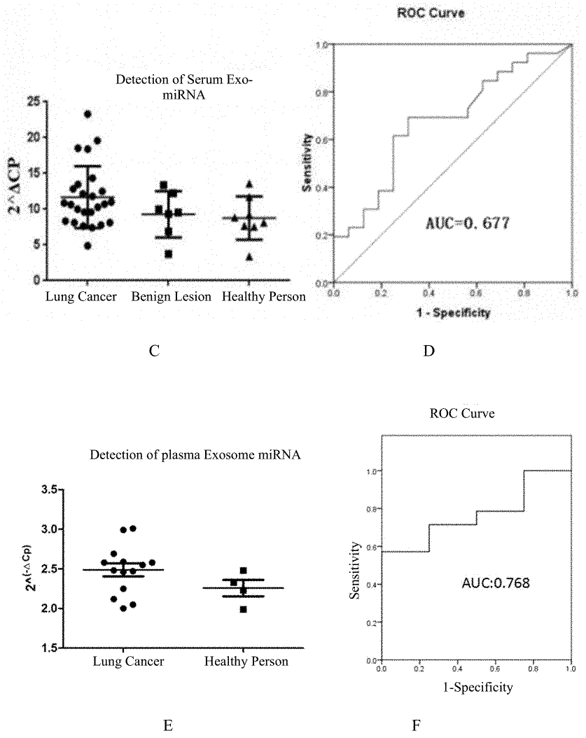

As shown in A and B of FIG. 8, the miRNAs in carcinoma and para-carcinoma tissue samples from 14 patients with lung cancer are detected, to detect the expression quantity CP values of the up-regulated marker miR-205 for miRNA and the down-regulated marker Let-7a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.821, showing a significant advantage.

As shown in C and D in FIG. 8, the Exo-miRNAs in serum samples from 25 patients with lung cancer in an early stage of Ia stage are detected in control group of 15 patients (healthy persons and persons with benign lesion), to detect the expression quantity CP values of up-regulated marker miR-126 for miRNA and down-regulated marker miR-152 for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.728, and when Cutoff value is taken as 55.98, the diagnose sensitivity is 64.0% and the specificity is 81.0%, showing a significant advantage.

As shown in E and F of FIG. 8, the up-regulated marker miR-126 for miRNA and the down-regulated marker miR-152 for miRNA correspond to plasma exosome microRNA in clinical sample in control group of samples from 16 patients with lung cancer in Ia stage and 3 healthy persons. The results show that plasma exosome microRNA has a certain degree of differentiation in control group of lung cancer and healthy persons.

As shown in G and H of FIG. 8, the Exo-miRNAs in urine samples from 25 patients with lung cancer in an early stage of Ia stage are detected in control group of 15 patients (healthy persons and persons with benign lesion), to detect the expression quantity CP values of the up-regulated marker miR-126 for miRNA and the down-regulated marker miR-152 for miRNA, and according to the CP values, the score of the relative expression quantity for miRNA is obtained. The detection results are analyzed through t detection by using SPSS17.0, to obtain P<0.05, indicating that the combined markers are significantly correlated with the prediction of early lung cancer,with AUC=0.719, and when Cutoff value is taken as 28.2230, the diagnose sensitivity is 65.9% and the specificity is 81.4%, showing a significant advantage.

(4) Detection Results of Combination of the Up-Regulated Marker miR-486-5p for miRNA and the miR-148a in a Clinical Sample

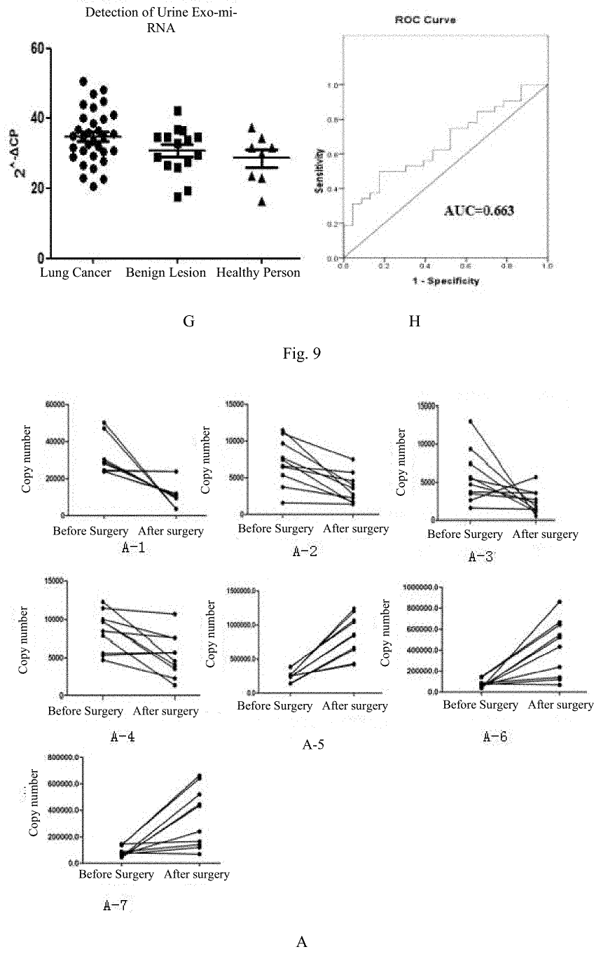

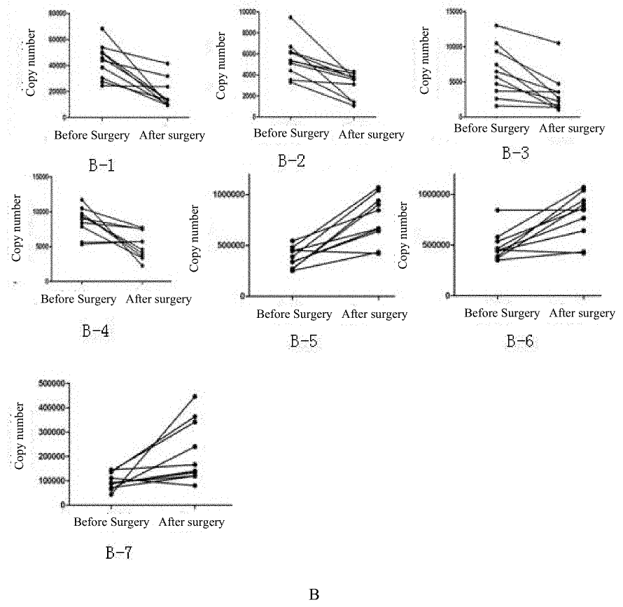

As shown in A and B of FIG. 9, the miRNAs in carcinoma and para-carcinoma tissue samples from 14 patients with lung cancer are detected, to detect the expression quantity CP values of the up-regulated marker miR-205 for miRNA and the down-regulated marker Let-7a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer, with AUC=0.837, showing a significant advantage.

As shown in C and D in FIG. 9, the Exo-miRNAs in serum samples from 25 patients with lung cancer in an early stage of Ia stage are detected in control group of 15 patients (healthy persons and persons with benign lesion), to detect the expression quantity CP values of the up-regulated marker miR-486-5p for miRNA and the down-regulated marker miR-148a for miRNA, and according to the CP values, multiple changes in relative expression quantity of the combined markers are calculated by using values of a relative quantitative formula (2^{-ΔΔCp}), thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.677, and when Cutoff value is taken as 9.98, the diagnose sensitivity is 61.5% and the specificity is 75.0%, showing a significant advantage.

As shown in E and F of FIG. 9, the combination of the miRNA markers miR-486-5p and the miR-148a correspond to plasma exosome microRNA in clinical sample in control group of samples from 16 patients with lung cancer in Ia stage and 3 healthy persons. The results show that plasma exosome microRNA has a certain degree of differentiation in control group of lung cancer and healthy persons.

As shown in G and H of FIG. 9, the Exo-miRNAs in urine samples from 25 patients with lung cancer in an early stage of Ia stage are detected in control group of 15 patients (healthy persons and persons with benign lesion), to detect the expression quantity CP values of the up-regulated marker miR-486-5p for miRNA and the down-regulated marker miR-148a for miRNA, and according to the CP values, the score of the relative expression quantity for miRNA is obtained. The detection results are analyzed through t detection by using SPSS17.0, to obtain P<0.05, indicating that the combined markers are significantly correlated with the prediction of early lung cancer, with AUC=0.663, and when Cutoff value is taken as 28.9214, the diagnose sensitivity is 65.2% and the specificity is 78.1%, showing a significant advantage.

8) miRNA Expression Levels and Differences Thereof in Body Fluid Exosome Before and After Surgery of Lung Cancer

(1) The body fluid (serum, plasma and urine) samples before surgery and the corresponding body fluid (serum, plasma and urine) samples after surgery from 10 patients with lung cancer and without any treatment diagnosed by hospital examination (comprising different stages, subtypes, gender and age groups) are collected, to detect the expression levels of miR-21, miRNA-205, miRNA-126, miRNA-486-5p, Let-7a, miR-152 and miR-148a.

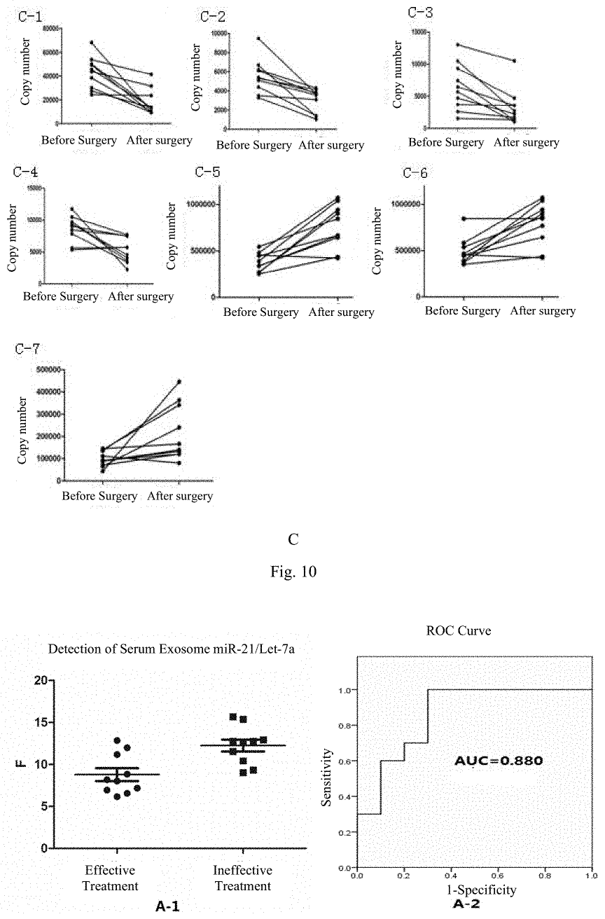

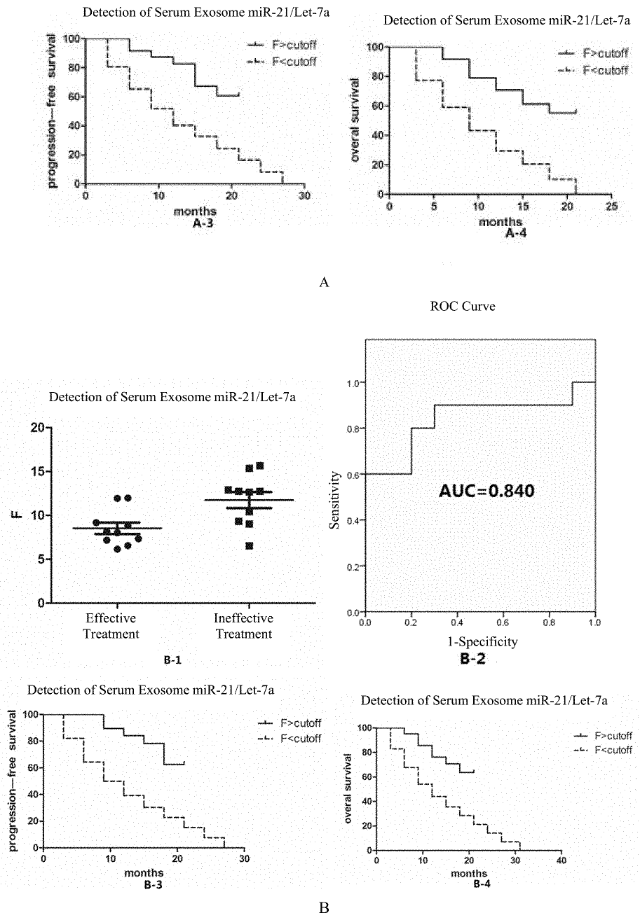

(2) The clinical samples of the up-regulated markers miR-21, miR-205, miR-126 and miR-486-5p for miRNA are detected, and the Exo-miRNAs in the body fluid (serum, plasma and urine) samples before surgery and the corresponding body fluid (serum, plasma and urine) samples after surgery from 10 patients with lung cancer and without any treatment are detected, to detect the expression quantity of the up-regulated markers miR-21, miR-205, miR-126 and miR-486-5p for miRNA. The detection results are analyzed through t detection by using SPSS17.0, to obtain P<0.05. The results that the differences of serum exosomes miR-21, miR-205, miR-126 and miR-486-5p in 1 week before and after surgery are statistically significant are shown in A of FIG. 10; the results that the differences of plasma exosomes miR-21, miR-205, miR-126 and miR-486-5p in 1 week before and after surgery are statistically significant are shown in B of FIG. 10; and the results that the differences of urine exosomes miR-21, miR-205, miR-126 and miR-486-5p in 1 week before and after surgery are statistically significant are shown in C of FIG. 10,showing that body fluid exosomes miR-21, miR-205, miR-126 and miR-486-5p are likely to become biochemical markers detected after surgery of lung cancer.

9) Detection Results of Exosome miRNA for Prognosis Evaluation

(1) The Exo-miRNAs in 20 samples from patients with lung cancer in an early stage of IA stage that could be treated surgically without distant metastasis and without major systemic disease are detected. The inclusion criteria of the samples is to be able to complete chemotherapy according to the predetermined scheme. The body fluid can be collected before and after chemotherapy, and the samples can be divided into 10 cases of effective treatment group and 10 cases of ineffective treatment group through pathological diagnosis. The expression levels of miR-21, miRNA-205, miRNA-126, miRNA-486-5p, Let-7a, miR-152 and miR-148a are detected. The relative expression quantity F=2^{-ΔΔcp} of a gene is calculated by adopting a relative quantitative method. The Exo-miRNAs of 10 body fluid samples (five of which are relapsed or metastasized within 2 years, resulting in death) with significantly different prognostic survival are detected, to detect the expression quantity of the up-regulated markers miR-21 and miR-205 and the down-regulated marker Let-7a for miRNA, and the expression quantity of a gene is F=

[symbol]

(2) Prognosis evaluation for lung cancer through detection by combination of exosomes miR-21 and Let-7a

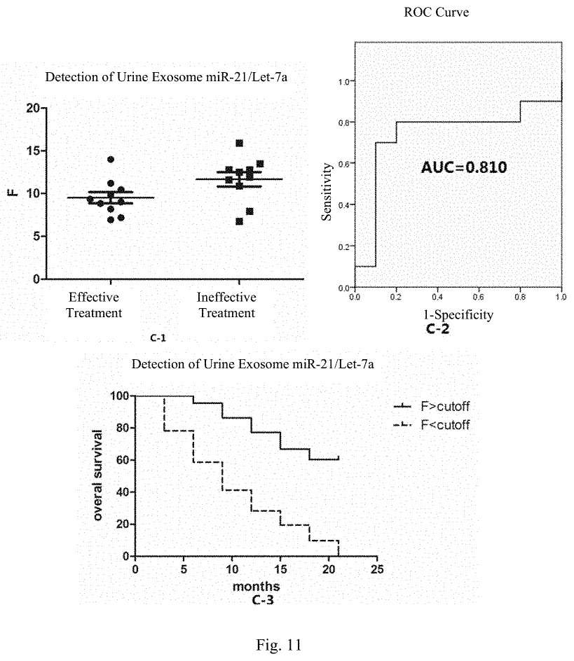

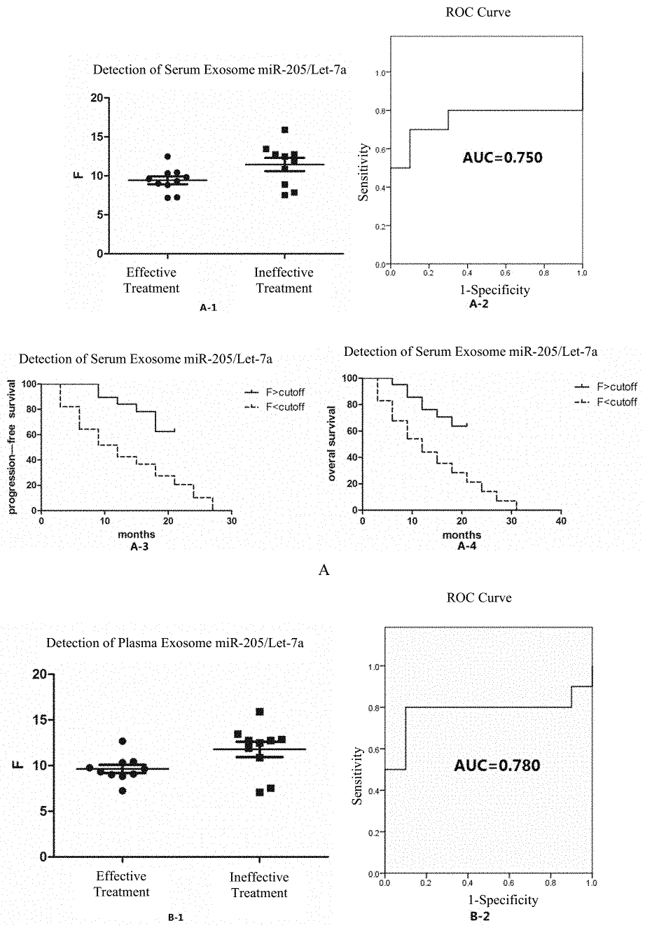

As shown in A of FIG. 11, 10 samples with effective treatment and 10 samples without effective treatment, the combined detection results of serum exosomes miR-21 and let-7a, and AUC=0.840, are significantly correlated with the therapeutic effect; andA Kaplan-Meier curve shows that the expression levels of combination of serum Exo-miR-21 and Let-7a are closely related to PFS of a patient, and the PFS of the patient in an F>cutoff group is longer (P<0.05).

As shown in B of FIG. 11, 10 samples with effective treatment and 10 samples without effective treatment, the combined detection results of plasma exosomes miR-21 and let-7a, and AUC=0.810, are significantly correlated with the therapeutic effect; and the Kaplan-Meier curve shows that the expression levels of combination of plasma Exo-miR-21 and Let-7a are closely related to the PFS of the patient, and the PFS of the patient in the F>cutoff group is longer (P<0.05).

As shown in C of FIG. 11, 10 samples with effective treatment and 10 samples without effective treatment, the combined detection results of urine exosomes miR-21 and let-7a, and AUC=0.750, are significantly correlated with the therapeutic effect; and the Kaplan-Meier curve shows that the expression levels of combination of urine Exo-miR-21 and Let-7a are closely related to the PFS of the patient, and the PFS of the patient in the F>cutoff group is longer (P<0.05).

(3) Prognosis evaluation for lung cancer through detection by combination of exosomes miR-205 and Let-7a

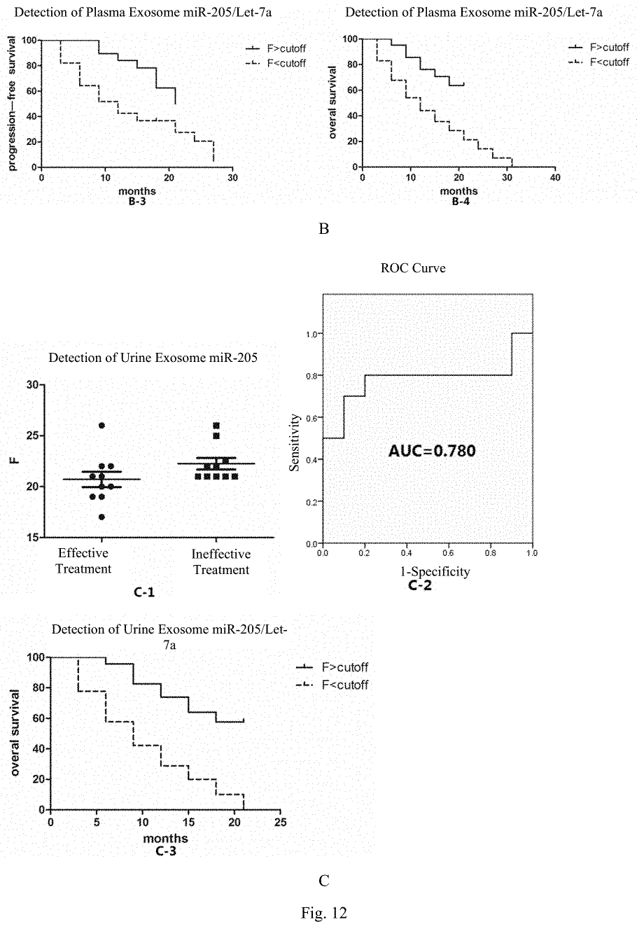

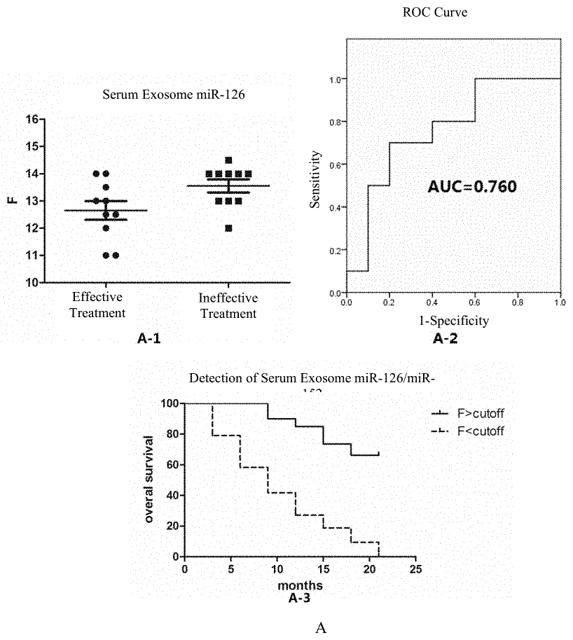

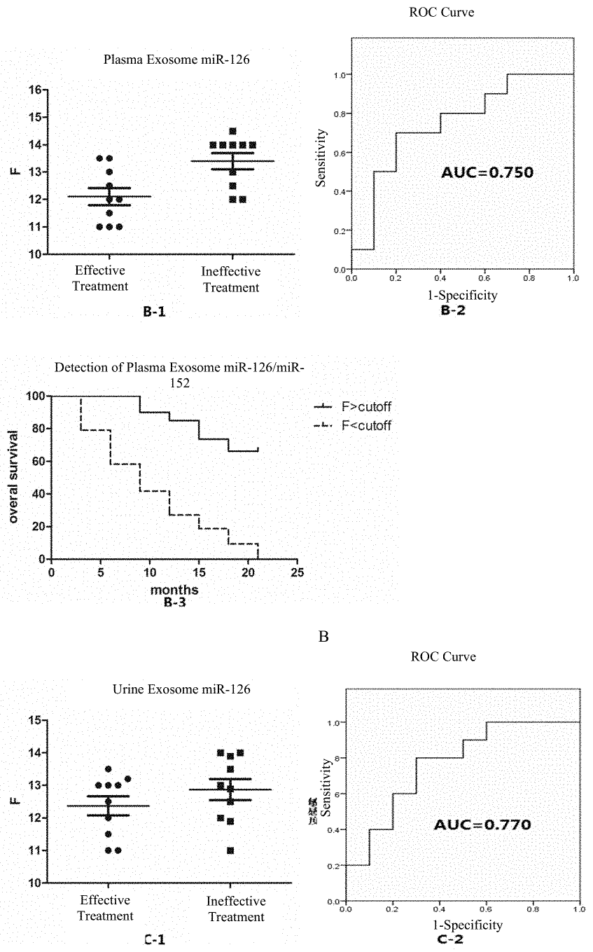

As shown in A of FIG. 12, 10 samples with effective treatment and 10 samples without effective treatment, the combined detection results of serum exosome miR-205 and let-7a, and AUC=0.750, are significantly correlated with the therapeutic effect; and the Kaplan-Meier curve shows that the expression levels of combination of serum Exo-miR-205 and Let-7a are closely related to the PFS of the patient, and the PFS of the patient in the F>cutoff group is longer (P<0.05).

As shown in B of FIG. 12, 10 samples with effective treatment and 10 samples without effective treatment, the combined detection results of plasma exosomes miR-205 and let-7a, and AUC=0.780, are significantly correlated with the therapeutic effect; and the Kaplan-Meier curve shows that the expression levels of combination of plasma Exo-miR-205 and Let-7a are closely related to the PFS of the patient, and the PFS of the patient in the F>cutoff group is longer (P<0.05).

10 samples with effective treatment and 10 samples without effective treatment, the combined detection results of urine exosomes miR-205 and let-7a, and AUC=0.780 are shown in C of FIG. 12. The Kaplan-Meier curve shows that the expression levels of combination of urine Exo-miR-205 and Let-7a are closely related to the PFS of the patient, and the PFS of the patient in the F>cutoff group is longer (P<0.05).

(4) Prognosis evaluation for lung cancer through detection by combination of exosomes miR-126 and miR-152

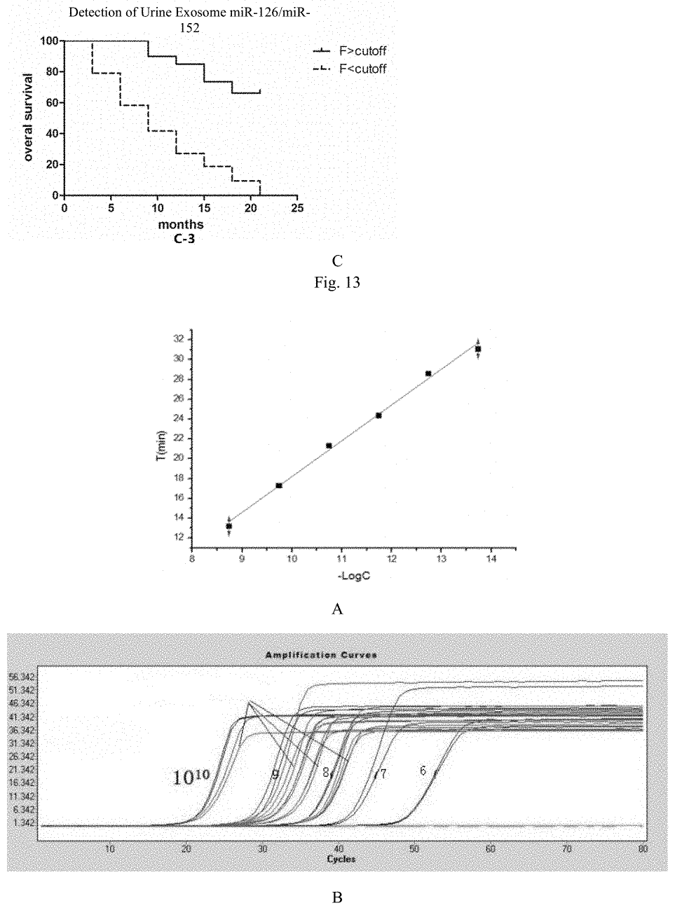

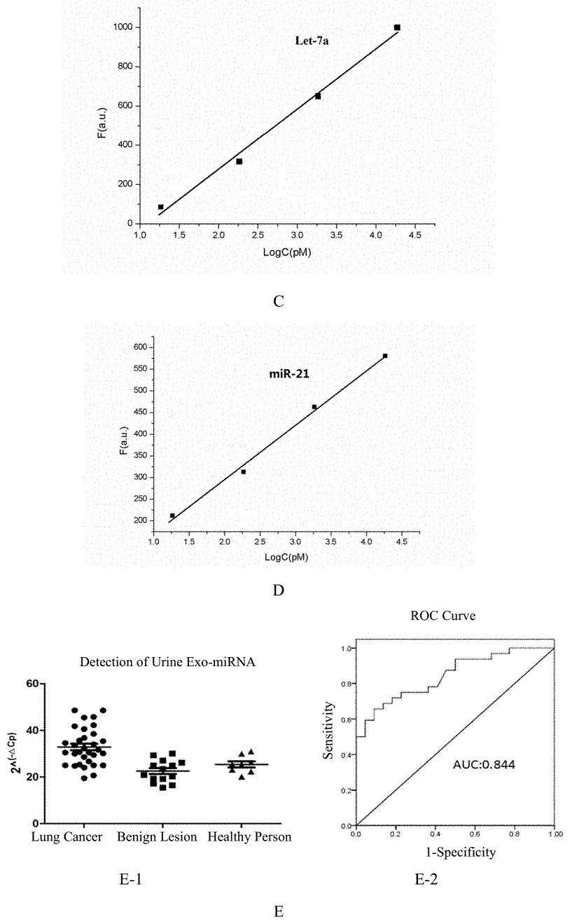

As shown in A of FIG. 13, 10 samples with effective treatment and 10 samples without effective treatment, the combined detection results of serum exosomes miR-126 and miR-152, and AUC=0.760, are significantly correlated with the therapeutic effect; and the Kaplan-Meier curve shows that the expression levels of combination of serum Exo-miR-126 and miR-152 are closely related to the PFS of the patient, and the PFS of the patient in the F>cutoff group is longer (P<0.05).

As shown in B of FIG. 13, 10 samples with effective treatment and 10 samples without effective treatment, the combined detection results of plasma exosomes miR-126 and miR-152, and AUC=0.750, are significantly correlated with the therapeutic effect; and the Kaplan-Meier curve shows that the expression levels of combination of plasma Exo-miR-126 and miR-152 are closely related to the PFS of the patient, and the PFS of the patient in the F>cutoff group is longer (P<0.05).

As shown in C of FIG. 13, 10 samples with effective treatment and 10 samples without effective treatment, the combined detection results of urine exosomes miR-126 and miR-152, and AUC=0.770, are significantly correlated with the therapeutic effect; and the Kaplan-Meier curve shows that the expression levels of combination of urine Exo-miR-126 and miR-152 are closely related to the PFS of the patient, and the PFS of the patient in the F>cutoff group is longer (P<0.05).

10) Detection Results of Exosome miRNA for Relapse Monitoring

(1) The body fluid samples (10 cases each) with the same pathological staging and significantly different prognosis survival in 2015-2016 are the first diagnosis of the primary lesion. Five patients are still alive 3 years after surgery, and five patients are relapsed or have lymph node metastasis or liver metastasis within 2 years after treatment, and are died within 2 years. The body fluid before and after surgery in 10 samples is collected to detect an expression level of the exosome miRNA, follow-up sampling detection is conducted every 3 months after surgery, and according to the expression conditions of the exosome miRNA from patients, prediction is conducted to judge the relapse or metastasis. The statistical analysis is conducted to evaluate the correlation between an expression level of exosome miRNA and imaging detection. The statistical analysis is conducted to evaluate the relationship between the expression level of the exosome miRNA and survival time from the patients.

(2) The expression quantity of a gene is F= [symbol] , and in 5 relapse samples, according to P=F in different time after treatment/F after chemotherapy, when P is more than 1.5, it is judged as relapsed, and P is less than or equal to 1.5, it is judged as not relapsed, and the P value judgment result is compared with the clinical evaluation results from 5 patients. (3) The miRNA markers are analyzed. The results that in 5 relapsed patients, the evaluation coincidence rate of the gene expression quantity of the exosome miR-21, miR-486-5p, miR-205, miR-126, miR-152, Let-7a or miR-148a is 100%, showing that the body fluid exosome miRNA is found earlier than clinical symptoms and signs are shown in Tables 6-10, and can be used for predicating the relapse or metastasis of lung cancer. The Kaplan-Meier survival and relapse analysis results that the gene expression quantity of the exosome miR-21, miR-486-5p, miR-205, miR-126, miR-152, Let-7a or miR-148a is significantly correlated with the survival time are shown in FIGS. 11, 12 and 13, and can be used for assessing the risk of the relapse.

TABLE 6
Body Fluid Exosome miRNA from No. 1 Relapsed Patient and Clinical Detection Results

| Patient | | | | | | |
|---|---|---|---|---|---|---|
| ID | | | | 3 months | 6 months | 9 months |
| 1 | Serum | Combination of miR-21 | P value | 0.989 | 1.51 | 1.55 |
| | exosome | and Let-7a | P value | | Relapsed | Relapsed |
| | | | judgment result | | | |
| | | Combination of miR- | P value | 0.55 | 0.79 | 1.50 |
| | | 205 and Let-7a | P value | | | Relapsed |
| | | | judgment result | | | |
| | | Combination of miR- | P value | 0.61 | 0.82 | 1.68 |
| | | 126 and miR-152 | P value | | | Relapsed |
| | | | judgment result | | | |
| | | Combination of miR- | P value | 0.44 | 0.81 | 1.54 |
| | | 486-5p and miR-148a | P value | | | Relapsed |
| | | | judgment result | | | |
| | Plasma | Combination of miR-21 | P value | 1.10 | 1.64 | 1.70 |
| | exosome | and Let-7a | P value | | Relapsed | Relapsed |
| | | | judgment result | | | |
| | | Combination of miR- | P value | 0.99 | 1.02 | 1.51 |
| | | 205 andLet-7a | P value | | | Relapsed |
| | | | judgment result | | | |
| | | Combination of miR- | P value | 0.82 | 1.06 | 1.59 |
| | | 126 and miR-152 | P value | | | Relapsed |
| | | | judgment result | | | |
| | | Combination of miR- | P value | 1.02 | 1.34 | 1.68 |
| | | 486-5p and miR-148a | P value | | | Relapsed |
| | | | judgment result | | | |
| | Urine | Combination of miR-21 | P value | 1.12 | 1.56 | 1.61 |
| | exosome | and Let-7a | P value | | Relapsed | Relapsed |
| | | | judgment result | | | |
| | | Combination of miR- | P value | 1.31 | 1.52 | 1.67 |
| | | 205 and Let-7a | P value | | Relapsed | Relapsed |
| | | | judgment result | | | |
| | | Combination of miR- | P value | 0.99 | 1.12 | 1.59 |
| | | 126 and miR-152 | P value | | | Relapsed |
| | | | judgment result | | | |
| | | Combination of miR- | P value | 0.87 | 1.03 | 1.63 |
| | | 486-5p and miR-148a | P value | | | Relapsed |
| | | | judgment result | | | |

| | |
|---|---|
| Clinical evaluation result | Relapsed |

TABLE 7
Body Fluid Exosome miRNA from No. 2 Relapsed Patient and Clinical Detection Results

| Patient | | | | | | | | |
|---|---|---|---|---|---|---|---|---|
| ID | | | | 3 months | 6 months | 9 months | 12 months | 15 months |
| 2 | Serum | Combination | P value | 0.232 | 0.43 | 0.93 | 1.62 | 1.88 |
| | exosome | of miR-21 | P value | | | | Relapsed | Relapsed |
| | | and Let-7a | judgment result | | | | | |
| | | Combination | P value | 0.25 | 0.29 | 0.48 | 0.64 | 1.50 |
| | | of miR-205 | P value | | | | | Relapsed |
| | | and Let-7a | judgment result | | | | | |
| | | Combination | P value | 0.11 | 0.32 | 0.39 | 0.70 | 1.61 |
| | | of miR-126 | P value | | | | | Relapsed |
| | | and miR-152 | judgment result | | | | | |
| | | Combination | P value | 0.36 | 0.61 | 0.822 | 1.30 | 1.51 |
| | | of miR-486- | P value | | | | | Relapsed |
| | | 5p and miR-148a | judgment result | | | | | |
| | Plasma | Combination | P value | 0.15 | 0.35 | 0.89 | 1.51 | 1.63 |
| | exosome | of miR-21 | P value | | | | Relapsed | Relapsed |
| | | and Let-7a | judgment result | | | | | |
| | | Combination | P value | 0.27 | 0.32 | 0.59 | 0.87 | 1.51 |
| | | of miR-205 | P value | | | | | Relapsed |
| | | and Let-7a | judgment result | | | | | |
| | | Combination | P value | 0.29 | 0.48 | 0.67 | 0.93 | 1.52 |
| | | of miR-126 | P value | | | | | Relapsed |
| | | and miR-152 | judgment result | | | | | |
| | | Combination | P value | 0.48 | 0.89 | 1.03 | 1.49 | 1.63 |
| | | of miR-486- | P value | | | | | Relapsed |
| | | 5p and miR-148a | judgment result | | | | | |
| | Urine | Combination | P value | 0.39 | 0.68 | 1.10 | 1.59 | 1.91 |
| | exosome | of miR-21 | P value | | | | Relapsed | Relapsed |
| | | and Let-7a | judgment result | | | | | |
| | | Combination | P value | 0.47 | 0.71 | 0.93 | 1.31 | 1.54 |
| | | of miR-205 | P value | | | | | Relapsed |
| | | and Let-7a | judgment result | | | | | |
| | | Combination | P value | 0.14 | 0.39 | 0.68 | 0.91 | 1.51 |
| | | of miR-126 | P value | | | | | Relapsed |
| | | and miR-152 | judgment result | | | | | |
| | | Combination | P value | 0.42 | 0.79 | 0.93 | 1.49 | 1.57 |
| | | of miR-486- | P value | | | | | Relapsed |
| | | 5p and miR-148a | judgment result | | | | | |

| | |
|---|---|
| Clinical evaluation result | Relapsed |

TABLE 8
Body Fluid Exosome miRNA from No. 3 Relapsed Patient and Clinical Detection Results

| Patient | | | | | | |
|---|---|---|---|---|---|---|
| ID | | | | 3 months | 6 months | 9 months |
| 3 | Serum | Combination of miR-21 | P value | 0.66 | 1.68 | 1.75 |
| | exosome | and Let-7a | P value | | Relapsed | Relapsed |
| | | | judgment result | | | |
| | | Combination of miR-205 | P value | 0.43 | 0.81 | 1.52 |
| | | and Let-7a | P value | | | Relapsed |
| | | | judgment result | | | |
| | | Combination of miR-126 | P value | 0.39 | 0.74 | 1.63 |
| | | and miR-152 | P value | | | Relapsed |
| | | | judgment result | | | |
| | | Combination of miR-486- | P value | 0.44 | 0.79 | 1.51 |
| | | 5p and miR-148a | P value | | | Relapsed |
| | | | judgment result | | | |
| | Plasma | Combination of miR-21 | P value | 0.51 | 1.39 | 1.62 |
| | exosome | and Let-7a | P value | | | Relapsed |
| | | | judgment result | | | |
| | | Combination of miR-205 | P value | 0.69 | 1.40 | 1.50 |
| | | and Let-7a | P value | | | Relapsed |
| | | | judgment result | | | |
| | | Combination of miR-126 | P value | 0.49 | 0.97 | 1.51 |
| | | and miR-152 | P value | | | Relapsed |
| | | | judgment result | | | |
| | | Combination of miR-486- | P value | 0.71 | 1.41 | 1.66 |
| | | 5p and miR-148a | P value | | | Relapsed |
| | | | judgment result | | | |
| | Urine | Combination of miR-21 | P value | 0.29 | 0.77 | 1.51 |
| | exosome | and Let-7a | P value judgment | | | Relapsed |
| | | | result | | | |
| | | Combination of miR-205 | P value | 0.41 | 1.06 | 1.63 |
| | | and Let-7a | P value | | | |
| | | | judgment result | | | |
| | | Combination of miR-126 | P value | 0.51 | 0.93 | 1.51 |
| | | and miR-152 | P value | | | Relapsed |
| | | | judgment result | | | |
| | | Combination of miR-486- | P value | 0.48 | 1.03 | 1.55 |
| | | 5p and miR-148a | P value | | | Relapsed |
| | | | judgment result | | | |

| | |
|---|---|
| Clinical evaluation result | Relapsed |

TABLE 9
body fluid Exosome miRNA from No. 4 Relapsed Patient and Clinical Detection Results

| Patient | | | | | | | |
|---|---|---|---|---|---|---|---|
| ID | | | | 3 months | 6 months | 9 months | 12 months |
| 4 | Serum | Combination of | P value | 0.31 | 0.74 | 1.68 | 1.70 |
| | exosome | miR-21 and Let-7a | P value | | | Relapsed | Relapsed |
| | | | judgment result | | | | |
| | | Combination of | P value | 0.36 | 0.47 | 0.91 | 1.53 |
| | | miR-205 and Let-7a | P value | | | | Relapsed |
| | | | judgment result | | | | |
| | | Combination of | P value | 0.25 | 0.43 | 0.89 | 1.50 |
| | | miR-126 and miR-152 | P value | | | | Relapsed |
| | | | judgment result | | | | |
| | | Combination of | P value | 0.43 | 0.65 | 0.94 | 1.60 |
| | | miR-486-5p and | P value | | | | Relapsed |
| | | miR-148a | judgment result | | | | |
| | Plasma | Combination of | P value | 0.32 | 0.51 | 1.34 | 1.59 |
| | exosome | miR-21 and Let-7a | P value | | | | Relapsed |
| | | | judgment result | | | | |
| | | Combination of | P value | 0.42 | 0.81 | 1.40 | 1.62 |
| | | miR-205 and Let-7a | P value | | | | Relapsed |
| | | | judgment result | | | | |
| | | Combination of | P value | 0.33 | 0.56 | 0.89 | 1.50 |
| | | miR-126 and miR-152 | P value | | | | Relapsed |
| | | | judgment result | | | | |
| | | Combination of | P value | 0.61 | 0.99 | 1.39 | 1.59 |
| | | miR-486-5p and | P value | | | | Relapsed |
| | | miR-148a | judgment result | | | | |
| | Urine | Combination of | P value | 0.38 | 0.89 | 1.23 | 1.69 |
| | exosome | miR-21 and Let-7a | P value | | | | Relapsed |
| | | | judgment result | | | | |
| | | Combination of | P value | 0.59 | 0.99 | 1.41 | 1.50 |
| | | miR-205 and Let-7a | P value | | | | Relapsed |
| | | | judgment result | | | | |
| | | Combination of | P value | 0.39 | 0.76 | 1.03 | 1.62 |
| | | miR-126 and miR-152 | P value | | | | Relapsed |
| | | | judgment result | | | | |
| | | Combination of | P value | 0.51 | 0.85 | 1.31 | 1.50 |
| | | miR-486-5p and | P value | | | | Relapsed |
| | | miR-148a | judgment result | | | | |

| | |
|---|---|
| Clinical evaluation result | Relapsed |

TABLE 10
Body Fluid Exosome miRNA from No. 5 Relapsed Patient and Clinical Detection Results

| Patient | | | | | | | | | |
|---|---|---|---|---|---|---|---|---|---|
| ID | | | | 3 months | 6 months | 9 months | 12 months | 15 months | 18 months |
| 5 | Serum | Combination | P value | 0.19 | 0.27 | 0.64 | 1.21 | 1.52 | 1.67 |
| | exosome | of miR-21 | P value | | | | | Relapsed | Relapsed |
| | | and Let-7a | judgment result | | | | | | |
| | | Combination | P value | 0.21 | 0.32 | 0.66 | 1.05 | 1.33 | 1.59 |
| | | of miR-205 | P value | | | | | | Relapsed |
| | | and Let-7a | judgment result | | | | | | |
| | | Combination | P value | 0.29 | 0.45 | 0.74 | 1.37 | 1.61 | 1.60 |
| | | of miR-126 | P value | | | | | Relapsed | Relapsed |
| | | and miR-152 | judgment result | | | | | | |
| | | Combination | P value | 0.32 | 0.61 | 0.91 | 1.46 | 1.52 | 1.61 |
| | | of miR-486- | P value | | | | | Relapsed | Relapsed |
| | | 5p and miR-148a | judgment result | | | | | | |
| | Plasma | Combination | P value | 0.32 | 0.71 | 1.01 | 1.33 | 1.59 | 1.70 |
| | exosome | of miR-21 | P value | | | | | Relapsed | Relapsed |
| | | and Let-7a | judgment result | | | | | | |
| | | Combination | P value | 0.37 | 0.89 | 0.99 | 1.28 | 1.49 | 1.51 |
| | | of miR-205 | P value | | | | | | Relapsed |
| | | and Let-7a | judgment result | | | | | | |
| | | Combination | P value | 0.45 | 0.90 | 1.37 | 1.42 | 1.60 | 1.67 |
| | | of miR-126 | P value | | | | | Relapsed | Relapsed |
| | | and miR-152 | judgment result | | | | | | |
| | | Combination | P value | 0.31 | 0.73 | 0.99 | 1.31 | 1.56 | 1.60 |
| | | of miR-486- | P value | | | | | Relapsed | Relapsed |
| | | 5p and miR-148a | judgment result | | | | | | |
| | Urine | Combination | P value | 0.22 | 0.69 | 0.81 | 1.35 | 1.62 | 1.70 |
| | exosome | of miR-21 | P value | | | | | Relapsed | Relapsed |
| | | and Let-7a | judgment result | | | | | | |
| | | Combination | P value | 0.49 | 0.81 | 1.31 | 1.12 | 1.44 | 1.50 |
| | | of miR-205 | P value | | | | | | Relapsed |
| | | and Let-7a | judgment result | | | | | | |
| | | Combination | P value | 0.35 | 0.65 | 0.92 | 1.29 | 1.50 | 1.59 |
| | | of miR-126 | P value | | | | | Relapsed | Relapsed |
| | | and miR-152 | judgment result | | | | | | |
| | | Combination | P value | 0.61 | 0.98 | 1.16 | 1.31 | 1.59 | 1.61 |
| | | of miR-486- | P value | | | | | Relapsed | Relapsed |
| | | 5p and miR-148a | judgment result | | | | | | |

| | |
|---|---|
| Clinical evaluation result | Relapsed |

### Embodiment 2

I. Isothermal Amplification-Based One-Step Detection Kit for miRNA

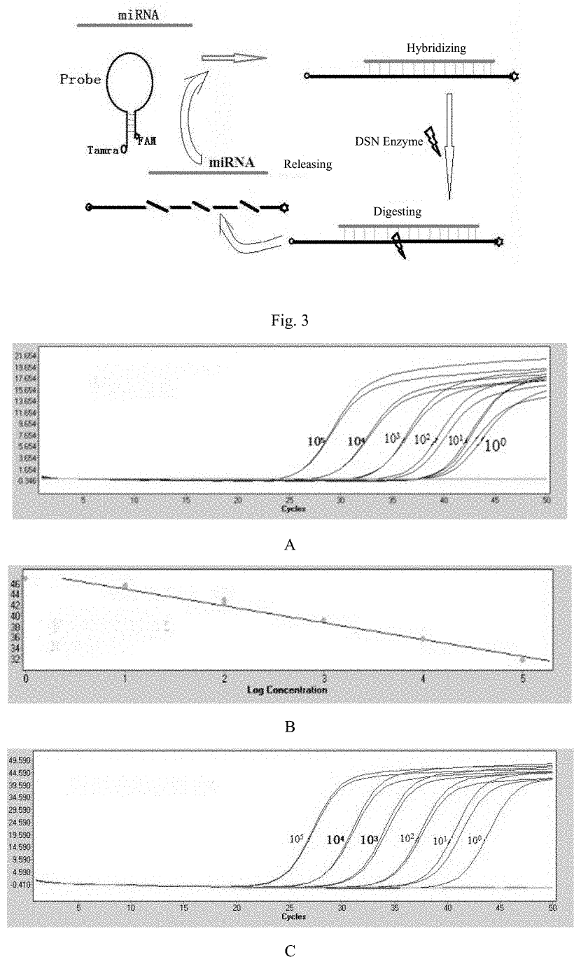

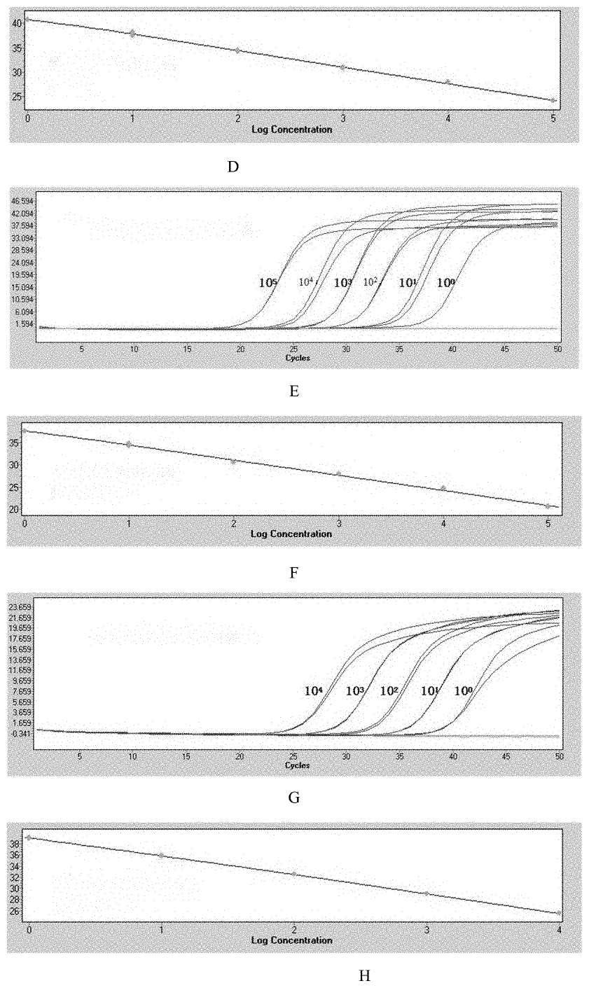

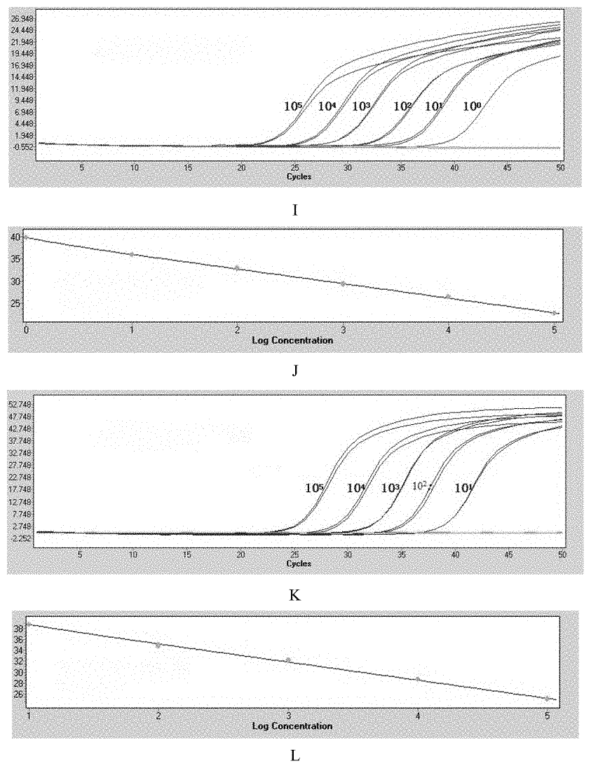

### 1. An Amplification Principle of EXPAR One-Step Detection System is Shown in FIG. 2.

1) Under the isothermal condition, through an EXPAR technique, no reverse transcription is required to achieve EXPAR, and the PCR amplification system of a miRNA molecular marker is shown in Table 11:

TABLE 11
PCR Amplification System of miRNA Molecular Marker

| Component | Final concentration | | Volume(μL) |
|---|---|---|---|
| Nt.BstNBI buffer | 0.5× | | 1 |
| Amplification template A | 0.1 | μmol/L | 1 |
| Amplification template B | 0.1 | μmol/L | 1 |
| dNTPs | 250 | μmol/L | 2 |
| RNase inhibitor | 0.8 | UμL⁻¹ | 0.4 |
| ThermoPol buffer | 1× | | 2 |
| Nt.BstNBI | 0.4 | UμL⁻¹ | 0.8 |
| Vent (exo-) DNA enzyme | 0.05 | UμL⁻¹ | 0.5 |
| SYBR Green I | 1× | | 1 |
| Template | | | 2 |
| Nuclease-free water | | | Watering to 20 μL |

The amplification conditions are shown in Table 12:

TABLE 12
PCR Reaction Thermal Cycling Condition

| Amplification | Inactivation | Cooling |
|---|---|---|
| 55° C., 30 sec signal 60 cycles | 85° C., 5 min | 50° C., 30 sec |

2) The Amplification Principle of EXPAR One-Step Detection Results for Let-7a is Shown in FIG. 3.

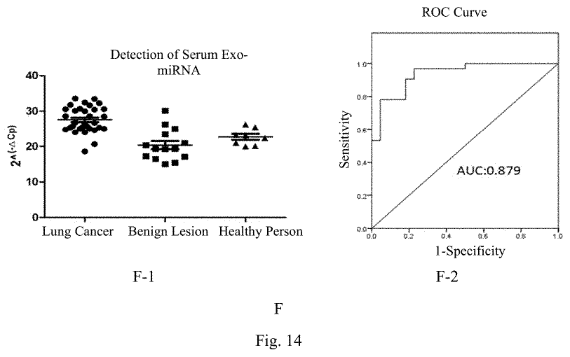

As shown in A of FIG. 14, the Let-7a standard is detected by adopting an EXPAR one-step method, a linear equation of the standard is POI=−17.9319−3.61353Log^{C(m)}, R²>0.99, the lower detection limit reaches 10⁵ copy/μL and the linear range is 5. As shown in B of FIG. 14, 10 clinical samples are detected, and the sample POI value falls within the linear range of 10⁷ to 10¹⁰ copies of the standard, showing that the EXPAR one-step method can be used for detecting the sample.

3) Advantages of the Present Invention

The differential amplification primer and enzymatic reagent are used to achieve the one-step detection for miRNA, a linear equation of the standard is POI=−17.9319−3.61353Log^{C(m)}, R²>0.99, the lower detection limit reaches 10⁵ copy/μL, the linear range is 5, the fluorescence signals are collected every 30 sec, and the reaction can be ended within 30 min. As shown in other similar EXPAR methods (literature: Guo-lei Wang and Chun-yang Zhang, Sensitive Detection of MicroRNAs with Hairpin Probe-Based Circular Exponential Amplification Detection. Anal. Chem. 2012, 84, 7037-7042), the lower detection limit is 10⁶ copy/μL, the linear range is 4, the reaction system operation is relatively complex, three systems such as A, B and C are required to be prepared and added in a reaction process one by one, and the reaction time is longer, about 100 min. The invention has a wider linear range, faster reaction time, better lower detection limit, and better amplification efficiency, and the isothermal amplification is more suitable for POCT field.

### 2. One-Step Detection System of Isothermal Linear Amplification

TABLE 13
Optimized PCR Reaction System

| | | |
|---|---|---|
| Component | Final concentration | Volume(μL) |

| | | |
|---|---|---|
| DSN buffer | 10× | 2 |

| | | | |
|---|---|---|---|
| DSN | 0.4 | UμL⁻¹ | 0.8 |
| probe | 0.5 | μM | 0.5 |
| RNase inhibitor | 0.8 | UμL⁻¹ | 0.8 |
| Template | | | 2 μL |
| Nuclease-free water | | | Watering to 20 μL |

TABLE 14
PCR Reaction Thermal Cycling Condition

| Amplification | Inactivation | Cooling | |
|---|---|---|---|
| 50° C., 120 min | 85° C., 5 min | 50° C., 30 sec | Collection of |
| | | | terminal signals |

Notes:
a. When the fluorescence PCR reaction volume is different, each reagent should be proportionally adjusted;
b. When the used instruments are different, the reaction parameters should be appropriately adjusted; and
c. Selection of instrument detection channel:

During the fluorescence PCR reaction, the collection of fluorescence signals of a reaction tube in the used instrument should be set, and the selected fluorescence detection channel should be consistent with the fluorescent reporter group labeled by a probe. The specific setting method varies from instrument to instrument, so please refer to an instrument operation manual.

After the isothermal reaction is finished, a fluorescence value is measured by using a fluorescence spectrophotometer, and a standard curve is made. The results are shown in C of FIG. 14, and the linear equation of Let-7a standard is Fluorescence(a.u.)=−339.22+307.44Log^{C(m)}, R²>0.99, the lower detection limit can reach 10⁷ copy/μL; andas shown in D of FIG. 14, the linear equation of miR-21 standard is Fluorescence(a.u.)=−44.509+125.55Log^{C(m)}, R²>0.99, the lower detection limit can reach 10⁷ copy/μL, and the linear range is 4, showing that the one-step system of isothermal linear amplification can be used for detecting the miRNA.

TABLE 11
Let-7a Fluorescence Value

| Copy number | Fluorescence(a.u.) | C(pM) | log C |
|---|---|---|---|
| 10\hat{ }11 | 1000.42 | 18500 | 4.267171728 |
| 10\hat{ }10 | 644.785 | 1850 | 3.267171728 |
| 10\hat{ }9 | 315.4215 | 185 | 2.267171728 |
| 10\hat{ }8 | 85.4215 | 18.5 | 1.267171728 |

TABLE 12
miR-21 Fluorescence Value

| Copy number | Fluorescence(a.u.) | C(pM) | log C |
|---|---|---|---|
| 10\hat{ }11 | 580.4588 | 18500 | 4.267171728 |
| 10\hat{ }10 | 462.5006 | 1850 | 3.267171728 |
| 10\hat{ }9 | 312.9769 | 185 | 2.267171728 |
| 10\hat{ }8 | 211.7934 | 18.5 | 1.267171728 |

II. Clinical Effect Evaluation of Isothermal Amplification-Based One-Step Detection Kit for miRNA

2) Sample Collection

The urine samples from persons with lung cancer diagnosed by hospital examination (comprising different stages, subtypes, gender and age groups), persons with pulmonary benign lesion and healthy persons are collected.

2) Extraction and Purification of Urine Exosome miRNA

The urine exosome is extracted by adopting a commercial product ExoQuick-TC for Tissue Culture Media and Urine kit in the SBI company (Art.No. EXOTC10A-1). The miRNA in the exosome is extracted and purified by adopting the commercial product miRNeasy mini kit in the QIAGEN company (Art.No. 217004) , and by using the Nano-Drop 2000, the RNA nucleic acid mass is measured, and the RNA concentration and the purity are recorded.

3) One-Step Detection System for miRNA

An isothermal amplification-based one-step detection system kit for miRNA in embodiment 2 is adopted. The Exo-miRNAs in serum samples from 50 patients with lung cancer in an early stage of Ia stage are detected in control group of 50 patients (healthy persons and persons with benign lesion), to detect expression quantity CP value of miR-152 and Let-7a, and according to the CP value, the relative expression quantity is calculated by using a relative quantitative formula.

4) Detection Results of Combination of miR-21 and Let-7a in a Clinical Sample of Lung Cancer

As shown in E of FIG. 14, the Exo-miRNAs in urine samples from 32 patients with lung cancer in an early stage of Ia stage are detected in control group of 22 patients (healthy persons and persons with benign lesion), to detect the expression quantity CP values of the up-regulated marker miR-21 for miRNA and the down-regulated marker Let-7a for miRNA, thus obtaining the score of the relative expression quantity for miRNA. The detection results are analyzed through t detection by using SPSS17.0, to obtain P<0.05, indicating that the combined markers are significantly correlated with the prediction of early lung cancer,with AUC=0.844, and when Cutoff value is taken as 19.878, the diagnose sensitivity is 87.5% and the specificity is 81.8%, showing a significant advantage.

As shown in F of FIG. 14, the Exo-miRNAs in serum samples from 56 patients with lung cancer in an early stage of Ia stage are detected in control group of 76 patients (healthy persons and persons with benign lesion), to detect the expression quantity CP values of the up-regulated marker miR-21 for miRNA and the down-regulated marker Let-7a for miRNA, and according to the CP values, the score of the relative expression quantity for miRNA is obtained. The detection results are analyzed through t detection by using SPSS17.0, to obtain P<0.05, indicating that the combined markers are significantly correlated with the prediction of early lung cancer, with AUC=0.879, and when Cutoff value is taken as 21.02, the diagnose sensitivity is 81.3% and the specificity is 81.8%, showing a significant advantage.

Finally, it should be noted that the above preferred embodiments are only used for describing the technical solution of the present invention rather than limiting the present invention. Although the present invention is already described in detail through the above preferred embodiments, those skilled in the art shall understand that various changes in form and detail can be made to the present invention without departing from the scope defined by claims of the present invention.

1. A use of an exosome-related microRNA molecular composition as a detection marker in preparing a kit for diagnosis and/or prediction of lung cancer, the exosome-related microRNA molecular composition comprising at least one up-regulated exosome microRNA molecule and at least one down-regulated exosome microRNA molecule.

2. The use according to claim 1, characterized in that the up-regulated exosome microRNA molecule is at least one of miR-21, miR-486-5p, miR-205 and miR-126, and the down-regulated microRNA molecule is at least one of miR-152, Let-7a and miR-148a.

3. The use according to claim 1, characterized in that the marker is a combination of miR-21 and Let-7a, a combination of miR-205 and Let-7a, a combination of miR-126 and miR-152 or a combination of miR-486-5p and miR-148a.

4. The use according to any one of claims 1, characterized in that the diagnosis and/or predication specifically include/includes lung cancer screening, identification of benign and malignant of early pulmonary nodules, auxiliary diagnosis of lung cancer, therapeutic effect evaluation of lung cancer, and prognosis evaluation or relapse monitoring of lung cancer.

5. The use according to any one of claims 1, characterized in that a detection sample of the kit is derived from body fluid or cell.

6. The use according to claim 5, characterized in that the body fluid comprises at least one of blood, serum, plasma, sputum, pleural effusion, pleural lavage fluid, urine and saliva.

7. A kit suitable for detecting a marker comprising.
a PCR platform-based two-step detection kit for miRNA or an isothermal amplification-based one-step detection kit for miRNA;
a PCR platform-based two-step detection kit for miRNA, including a specific stem-loop RT primer of the microRNA molecular marker, a PCR forward primer, a PCR universal reverse primer, a specific probe for detecting a microRNA molecular marker, and microRNA standards of serial gradient dilution concentrations; and
a loop of the neck of the specific stem-loop RT primer designed with a discontinuous complementary base pair TGCG and CGCA to form a key-like structure. and a short arm connected to the microRNA molecule through a ligase during a reverse transcription reaction.

8. (canceled)

9. (canceled)

10. (canceled)

11. The kit according to claim 7, wherein the one or more primers and probes of the microRNA molecular marker comprise RT-primer sequence of the miR-21 as shown in SEQ ID NO. 1, the PCR forward primer as shown in SEQ ID NO. 2, the PCR universal reverse primer as shown in SEQ ID NO. 3, and the specific probe nucleotide sequence as shown in SEQ ID NO. 4; the RT-primer sequence of the miR-486-5p as shown in SEQ ID NO. 5, the PCR forward primer as shown in SEQ ID NO. 6, the PCR universal reverse primer as shown in SEQ ID NO. 3, and the specific probe nucleotide sequence as shown in SEQ ID NO. 7; the RT-primer sequence of the miR-205 as shown in SEQ ID NO. 8, the PCR forward primer as shown in SEQ ID NO. 9, the PCR universal reverse primer as shown in SEQ ID NO. 3, and the specific probe nucleotide sequence as shown in SEQ ID NO. 10; the RT-primer sequence of the miR-126 as shown in SEQ ID NO. 11, the PCR forward primer as shown in SEQ ID NO. 12, the PCR universal reverse primer as shown in SEQ ID NO. 3, and the specific probe nucleotide sequence as shown in SEQ ID NO. 13; the RT-primer sequence of the let-7a as shown in SEQ ID NO. 14, the PCR forward primer as shown in SEQ ID NO. 15, the PCR universal reverse primer as shown in SEQ ID NO. 3, and the specific probe nucleotide sequence as shown in SEQ ID NO.16; the RT-primer sequence of the miR-152 as shown in SEQ ID NO. 17, the PCR forward primer as shown in SEQ ID NO. 18, the PCR universal reverse primer as shown in SEQ ID NO. 3, and the specific probe nucleotide sequence as shown in SEQ ID NO. 19; and the RT-primer sequence of the miR-148a as shown in SEQ ID NO. 20, the PCR forward primer as shown in SEQ ID NO. 21, the PCR universal reverse primer as shown in SEQ ID NO. 3, and the specific probe nucleotide sequence as shown in SEQ ID NO. 22.

12. The kit according to claim 7, further comprising miRNA molecular marker standards, wherein the miR-21 molecular marker standard is miR-21, diluted to a concentration of 10¹³ copy/μL, and gradiently diluted into a serial standard; the miR-486-5p molecular marker standard is miR-486-5p, diluted to a concentration of 10¹³ copy/μL, and gradiently diluted into a serial standard; the miR-205 molecular marker standard is miR-205, diluted to a concentration of 10¹³ copy/μL, and gradiently diluted into a serial standard; the miR-126 molecular marker standard is miR-126, diluted to a concentration of 10¹³ copy/μL, and gradiently diluted into a serial standard; the let-7a molecular marker standard is let-7a, diluted to a concentration of 10¹³ copy/μL, and gradiently diluted into a serial standard; the miR-152 molecular marker standard is miR-152, diluted to a concentration of 10¹³ copy/μL, and gradiently diluted into a serial standard; and the miR-148a molecular marker standard is miR-148a, diluted to a concentration of 10¹³ copy/μL, and gradiently diluted into a serial standard.

13. The kit according to claim 7, characterized in that the kit is an isothermal amplification-based one-step detection kit for miRNA, comprising specific amplification templates of microRNA molecules, Vent (exo-) DNA polymerase, nicking enzyme, duplex-specific nuclease and molecular hybridization probes.

14. The kit according to claim 13, further comprising one or more amplification template nucleotides and hybridization probes of the microRNA molecular marker, wherein the one or more amplification template nucleotides and hybridization probes of the microRNA molecular marker comprise: the first specific amplification template nucleotide of the miR-21 is as shown in SEQ ID NO. 23, the second amplification template nucleotide sequence of the miR-21 is as shown in SEQ ID NO. 24, and the hybridization probe sequence of the miR-21 is as shown in SEQ ID NO. 25; the first specific amplification template nucleotide of the miR-486-5p is as shown in SEQ ID NO. 26, the second amplification template nucleotide sequence of the miR-486-5p is as shown in SEQ ID NO. 27, and the hybridization probe sequence of the miR-486-5p is as shown in SEQ ID NO. 28; the first specific amplification template nucleotide of the miR-205 is as shown in SEQ ID NO. 29, the second amplification template nucleotide sequence of the miR-205 is as shown in SEQ ID NO. 30, and the hybridization probe sequence of the miR-205 is as shown in SEQ ID NO. 31; the first specific amplification template nucleotide of the miR-126 is as shown in SEQ ID NO. 32, the second amplification template nucleotide sequence of the miR-126 is as shown in SEQ ID NO. 33, and the hybridization probe sequence of the miR-126 is as shown in SEQ ID NO. 34; and the first specific amplification template nucleotide of the Let-7a is as shown in SEQ ID NO. 35, the second amplification template nucleotide sequence of the Let-7a is as shown in SEQ ID NO. 36, and the hybridization probe sequence of the Let-7a is as shown in SEQ ID NO. 37.

15. A method for diagnosis and/or prediction of individual lung cancer by using a detection kit comprising: PCR platform-based two-step detection kit for miRNA or an isothermal amplification-based one-step detection kit for miRNA; a PCR platform-based two-step detection kit for miRNA, including a specific stem-loop RT primer of the microRNA molecular marker, a PCR forward primer, a PCR universal reverse primer, a specific probe for detecting a microRNA molecular marker, and microRNA standards of serial gradient dilution concentrations; and a loop of the neck of the specific stem-loop RT primer designed with a discontinuous complementary base pair TGCG and CGCA to form a key-like structure, and a short arm connected to the microRNA molecule through a ligase during a reverse transcription reaction.

16. The method according to claim 15, characterized in that the diagnosis and/or predication specifically include/includes lung cancer screening, identification of benign and malignant of early pulmonary nodules, auxiliary diagnosis of lung cancer, therapeutic effect evaluation of lung cancer, and prognosis evaluation or relapse monitoring of lung cancer.

17. The method according to claim 15, characterized by comprising detecting a biological sample separated from an individual.

18. The method according to claim 17, wherein the biological sample is derived from body fluid or cell.

19. The method according to claim 18, wherein the body fluid comprises at least one of blood, serum, plasma, sputum, pleural effusion, pleural lavage fluid, urine and saliva.

20.-35. (canceled)
